(19) United States
(12) Patent Application Publication
(10) Pub. No.: US 2024/0119367 A1
(43) Pub. Date: Apr. 11, 2024

(54) MACHINE LEARNING BASED VOC DETECTION

(71) Applicants: George Mason University, Fairfax, VA (US); Oakland University, Rochester, MI (US)

(72) Inventors: Qiliang Li, Fairfax, VA (US); Pei Dong, Fairfax, VA (US); Xiaozhou Huang, Manassas, VA (US); Yaonian Li, Fairfax, VA (US); Zhe Wang, Rochester, MI (US)

(21) Appl. No.: 18/480,429

(22) Filed: Oct. 3, 2023

Related U.S. Application Data
(60) Provisional application No. 63/413,039, filed on Oct. 4, 2022.

(51) Int. Cl. G06N 20/00; G01N 27/416

(52) U.S. Cl. CPC G06N 20/00; G01N 27/4162; G01N 27/4168

(57) ABSTRACT

A machine-learning based VOC detection and identification method and an apparatus and system for implementing same.

### CROSS-REFERENCE TO RELATED APPLICATIONS

This application claims priority to U.S. Provisional Application No. 63/413,039, filed Oct. 4, 2022, the entirety of which is incorporated into this application by reference.

### STATEMENT OF GOVERNMENT SUPPORT

Certain aspects of the following disclosure were made with government support under grant number DE-AC02-06CH11357, awarded by the U.S. Department of Energy (DOE). The government has certain rights in the invention.

### BACKGROUND

Volatile organic compounds (VOCs) are a group of organic compounds characterized by low boiling points and facile evaporation at room temperature. Most VOCs, such as methanol, ethanol, and formaldehyde, threaten both environmental safety and human health. For instance, formaldehyde is known as one of the major chemical hazards existing in building materials and new furniture, which have long-term health effects and may cause cancer. VOCs are also known as biomarkers for specific diseases. For example, patients who were infected with Mycobacterium tuberculosis showed an increased level of cyclohexane and benzene derivatives from their body; 2-butanone, 1-propanol, isoprene, ethylbenzene, styrene and hexanal are biomarkers for lung cancer. These VOC biomarkers exist in human breath as well as human fluids. To have better monitoring for environmental safety and human health, it is important to have detectors for both liquid and gas VOCs.

Currently, VOCs can be detected and quantified by a series of techniques such as gas chromatography (GC), laser absorption spectrometry, and quartz crystal microbalance sensors. These analytical techniques have advantages in high sensitivity, high selectivity, multi-species detection, standoff detection, and maximizing information from the analyte. However, they are limited by high cost, low detection speed, and high complexity, which makes them unsuitable for early-stage diagnosis or frequent detections of harmful substances in the field.

Alternatively, VOCs can also be detected by several types of commercial detectors, mainly metal oxide sensors (MOS), photoionization detectors (PID), and electrochemical (EC) sensors. MOS detectors are less expensive, portable, and easy-to-use, but they suffer from low selectivity, cross-sensitivity, and calibration difficulties, which result in low reproducibility. PID detectors are more expensive but more sensitive than MOS detectors that usually can detect VOCs at ppb levels with a dynamic detection range (around 1 ppb to 1000 ppm). They are efficient and robust in most situations but not suitable for advanced applications especially when the detection environment is changing. EC detectors can quantify particular gases at the ppm level with low-power, high resolution, and excellent repeatability. However, EC detectors are limited by cross-sensitivity of other substances, short lifetime, narrow working temperature, and difficulties in determining baseline. Therefore, there is a need in the art for the development of advanced VOC detectors.

### SUMMARY

It is to be understood that both the following general description and the following detailed description are exemplary and explanatory only and are not restrictive. Methods, systems, and apparatuses for machine learning based VOC detection are described. A computing device may receive, from a sensor device, sensing data. The sending data may indicate one or more voltammograms associated with a sample. The sample may comprise an ionic liquid (IL), an aprotic solvent, and one or more unknown analytes. The computing device may determine, based on the sensing data, a plurality of features associated with the one or more voltammograms. The plurality of features may indicate shapes and redox peaks associated with the one or more voltammograms. The computing device may determine, based on the plurality of features, one or more linear discriminants associated with the one or more unknown analytes. The one or more linear discriminates may comprise one or more data points in a linear diagram. The computing device may classify, based on the one or more linear discriminants and one or more reference linear discriminants, the one or more unknown analytes. The one or more reference linear discriminates may comprise one or more reference data points in the linear diagram. The computing device may classify the one or more unknown analytes using a machine learning model. The machine learning model may be configured to determine the one or more reference discriminants based on one or more known analytes.

This summary is not intended to identify critical or essential features of the disclosure, but merely to summarize certain features and variations thereof. Other details and features will be described in the sections that follow.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1A is an exemplary system for machine learning based Volatile organic compounds (VOC) detection.

FIG. 1B is an exemplary system for machine learning based VOC detection.

FIG. 1C is an exemplary training method for machine learning based VOC detection.

FIG. 1D is an exemplary training module for machine learning based VOC detection.

FIG. 2A is an exemplary method for machine learning based VOC detection.

FIG. 2B is an exemplary plot of linear discriminant analysis (LDA) results with classification determination.

FIG. 3 is a diagram depicting a general structure of a disclosed sensor used to acquire cyclic voltammogram data.

FIG. 4A is a general schematic showing an exemplary method for detecting unknown analytes such as VOCs in a sample using cyclic voltammetry, linear discriminate analysis, and machine learning.

FIG. 4B is an exemplary plot of cyclic voltammetry generated using the disclosed method.

FIG. 4C is an exemplary plot generated from the cyclic voltammetry data plot shown in FIG. 4B.

FIG. 5 is a general schematic showing an exemplary method for detecting unknown analytes such as VOCs in a sample using cyclic voltammetry, linear discriminate analysis, and machine learning, and subsequent identification and classification of the unknown analytes.

FIG. 6 is a diagram showing the detection system for exemplary electrochemical studies for the IL/DMSO electrolyte.

FIG. 7 shows CV plots of pure IL with no analyte and IL containing 50 μL methanol, acquired from the detection system depicted in FIG. 6.

FIG. 8 shows CV plots of 10% IL/DMSO with no analyte and 10% IL/DMSO containing 50 μL methanol, acquired from the detection system depicted in FIG. 6.

FIG. 9 shows CV plots of 10% IL/DMSO at scan rates of 36, 49, 64, 81, 100 mV/s, acquired from the detection system depicted in FIG. 6.

FIG. 10 shows plots of reduction peak current density changes for electrolyte with different concentrations at scan rates of 36, 49, 64, 81, 100 mV/s, acquired from the detection system depicted in FIG. 6.

FIG. 11 shows a plot of the ratio of reduction peak current density (jₚᵣ) to oxidation peak current density (jₚₒ) for IL/DMSO at different concentrations. Data was collected at scan rate of 100 mV/s, acquired from the detection system depicted in FIG. 6.

FIG. 12A shows CV plots for DMSO relative to no analyte, acquired from the detection system depicted in FIG. 6.

FIG. 12B shows CV plots for methanol relative to no analyte, acquired from the detection system depicted in FIG. 6.

FIG. 12C shows CV plots for water relative to no analyte, acquired from the detection system depicted in FIG. 6.

FIG. 12D shows CV plots for ethanol relative to no analyte, acquired from the detection system depicted in FIG. 6.

FIG. 12E shows CV plots for acetone relative to no analyte, acquired from the detection system depicted in FIG. 6.

FIG. 12F shows CV plots for formaldehyde relative to no analyte, acquired from the detection system depicted in FIG. 6.

FIG. 12G shows CV plots for a mixture of VOCs relative to no analyte, acquired from the detection system depicted in FIG. 6.

FIG. 13 shows a general data acquisition and analysis flow chart for identifying unknown analytes in a sample using CV data and linear discriminant analysis (LDA), using data collected from the detection system depicted in FIG. 6.

FIG. 14 shows a plot of LDA results obtained for the DMSO control, methanol, water, ethanol, acetone, formaldehyde, and the VOC mixture, acquired according to the flow chart shown in FIG. 13, using data collected from the detection system depicted in FIG. 6.

FIG. 15A shows CV plots for electrolytes containing different amounts of 16% CH₂O/water solution, acquired from the detection system depicted in FIG. 6.

FIG. 15B shows a logarithmic plot corresponding to the CV plots shown in FIG. 15A for a 16% (w/v) CH₂O/water solution at a volume from 0 to 50 μL.

FIG. 15C shows a logarithmic plot corresponding to the CV plots shown in FIG. 15A for a 1.6% (w/v) CH₂O/water solution at a volume from 0 to 50 μL.

FIG. 15D shows a plot of LDA results for an exemplary CH₂O/water solution quantification, using the detection system depicted in FIG. 6.

FIG. 15E shows a plot providing linear correlations between impedance and volume for a 16% CH₂O water solution. The concentration of CH₂O/water solution is percentage mass/volume (w/v). These data were acquired from the detection system depicted in FIG. 6.

FIG. 15F shows a plot providing linear correlations between impedance and volume for a 1.6% CH₂O water solution. The concentration of CH₂O/water solution is percentage mass/volume (w/v). These data were acquired from the detection system depicted in FIG. 6.

FIG. 16 is an exemplary system for machine learning based Volatile organic compounds (VOC) detection.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memrsistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general-purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

This detailed description may refer to a given entity performing some action. It may be understood that this language may in some cases mean that a system (e.g., a computer) owned and/or controlled by the given entity is actually performing the action.

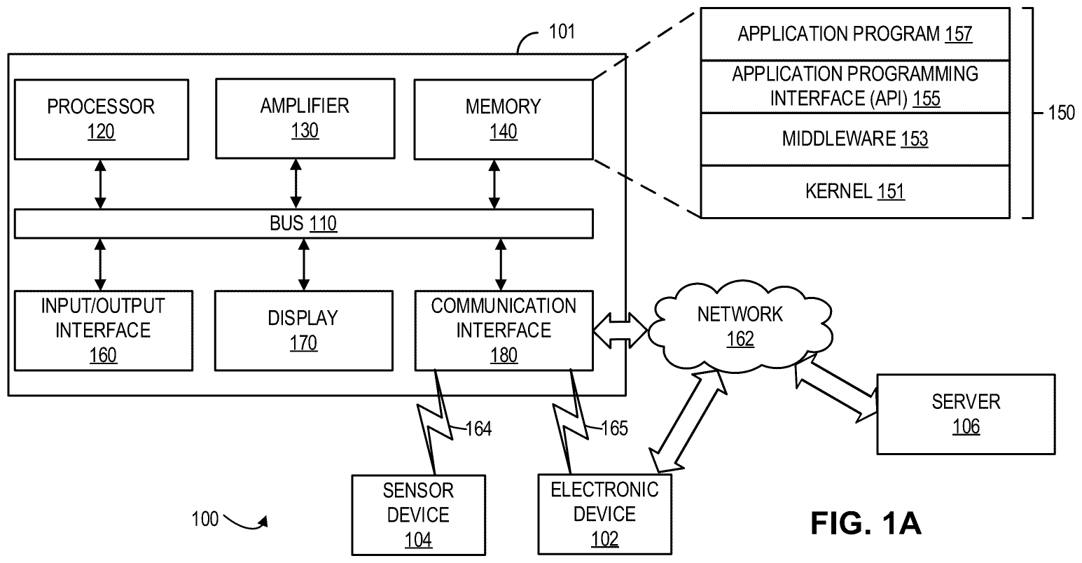

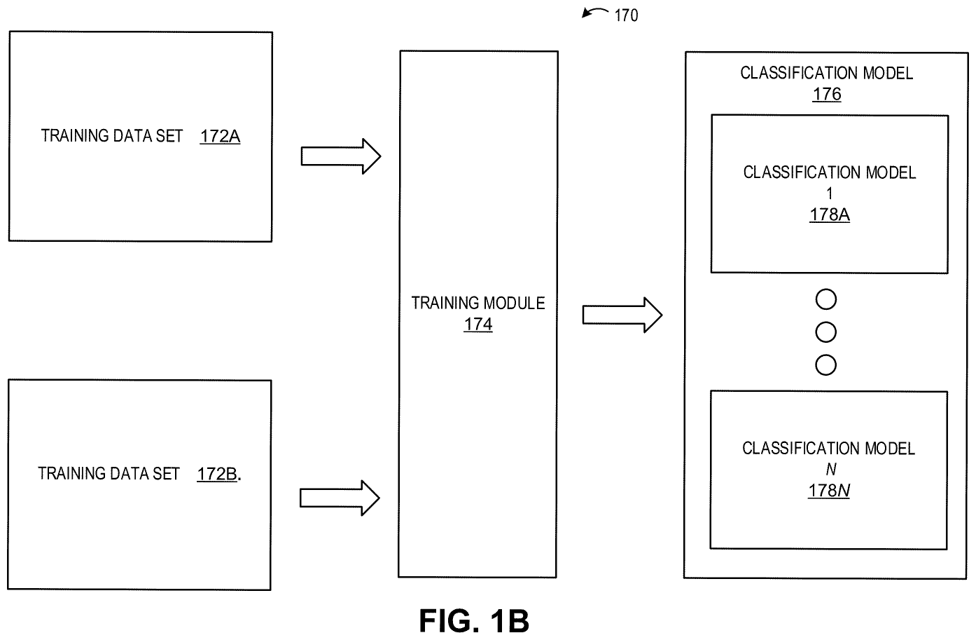

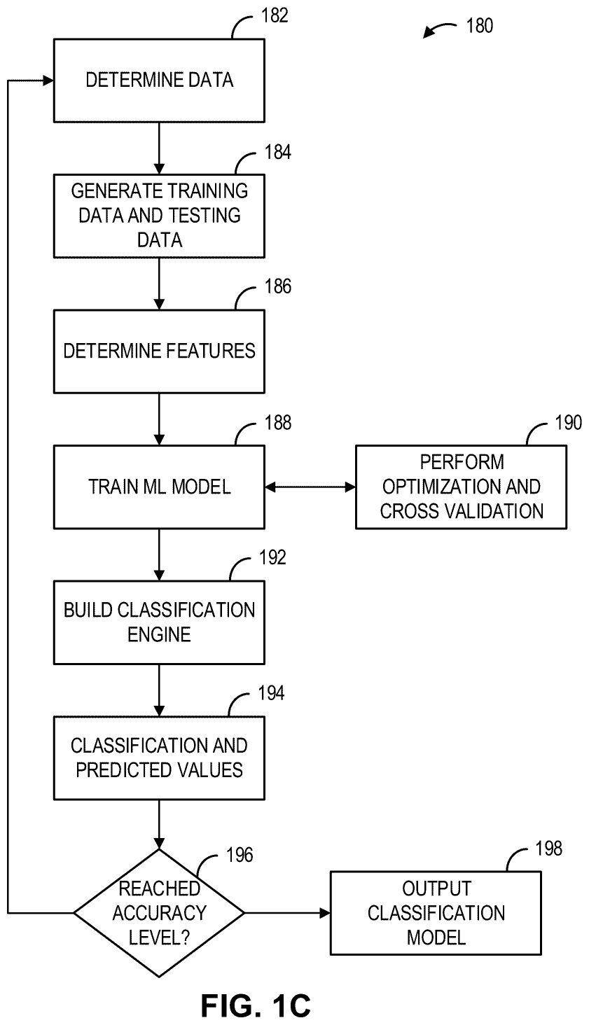

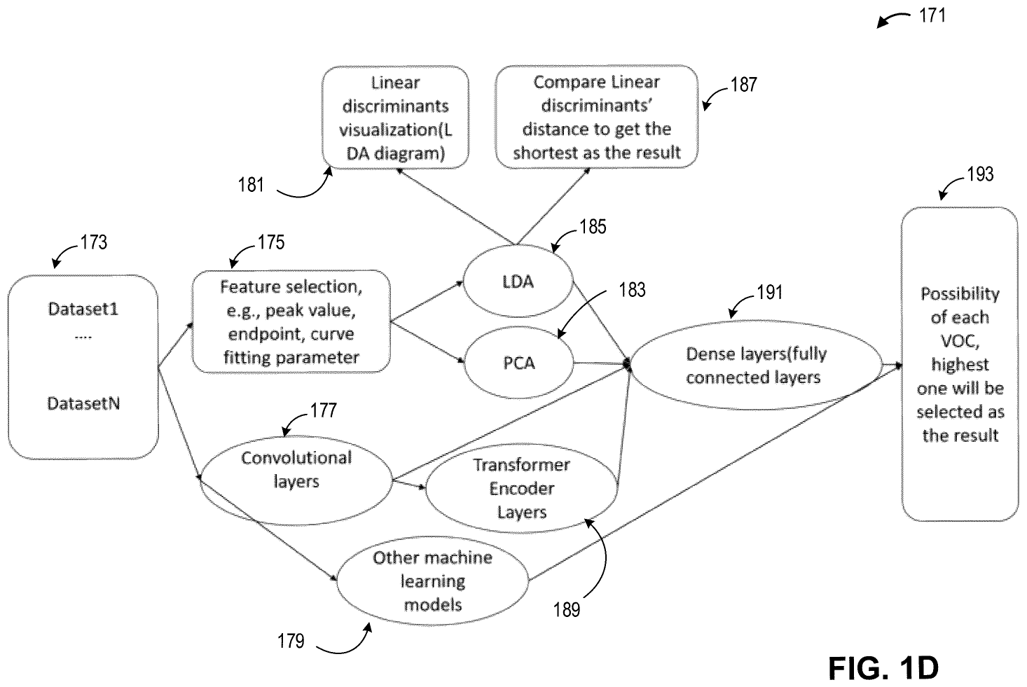

FIG. 1A shows an exemplary system 100 for machine learning based Volatile organic compounds (VOC) detection, where the methods, apparatuses, and systems described herein may be implemented according to various embodiments. Referring to FIG. 1A, a computing device 101 in the system 100 is disclosed according to various exemplary embodiments. The computing device 101 may include a bus 110, a processor 120, an amplifier 130, a memory 140, an input/output interface 160, a display 170, and a communication interface 180. In a certain exemplary embodiment, the computing device 101 may omit at least one of the aforementioned constitutional elements or may additionally include other constitutional elements. The computing device 101 may comprise a microcomputer, a miniature computer, a single board computer, a microcontroller, or a circuit board. In an embodiment, the computing device 101 may be, for example, a laptop, a desktop computer, a server, a electrochemical workstation, a electrochemical detector, an air quality detector, an air purifier, a mobile phone, a smart phone, a tablet computer, and the like.

The computing device 101 may be configured to process the electrochemical signals or sensing data received from another device such as a sensor device 104. The computing device 101 may receive the electrochemical signals or sensing data using a wireless connection or a wired connection. In an embodiment, the computing device 101 may forward the received electrochemical signals or sensing data to an external electronic device 102 and/or a server 106 for further processing. The external electronic device 102 and/or the server 106 may process the electrochemical signals or sensing data and transmit the processed data to the computing device 101 or other devices related to the sensor device 104.

The bus 110 may include a circuit for connecting the aforementioned constitutional elements 110 to 180 to each other and for delivering communication (e.g., a control message and/or data) between the aforementioned constitutional elements. For instance, the bus 110 may be designed to send the signals or sensor data from the processor 120 to the communication interface 180 in order to further transmit the signals or sensor data to an external device such as the electronic device 102 and/or a server 106.

The processor 120 may include one or more of a Microcontroller Unit (MCU), a Central Processing Unit (CPU), an Application Processor (AP), and a Communication Processor (CP). The processor 120 may control, for example, at least one of the other constitutional elements of the computing device 101 and/or may execute an arithmetic operation or data processing for communication. The processing (or controlling) operation of the processor 120 according to various embodiments is described in detail with reference to the following drawings. The processor 120 may include an on-chip analog-to-digital converter (ADC) for converting the amplified voltage signal, received from the amplifier 130 (described below), from an analog signal to a digital signal. The processor 120 may be used to process the digital signal, converted from electrochemical signals or sensing data received from the sensor device 104 or an electrochemical workstation. The processor 120 may then send the processed data, including classification of VOCs, to the communication interface 180 (which may include a Bluetooth module as shown below) using a Universal Asynchronous Receiver/Transmitter (UART), wherein the communication interface 180 may further transmit the processed data to an external electronic device 102, such as the electronic device 102, or the server 106.

In an embodiment, the processor 120 may be configured to perform machine learning based volatile organic compound (VOC) detection. For example, the processor 120 may receive sensing data from the sensor device 104. The sensing data may indicate one or more voltammograms associated with a sample that comprises an ionic liquid (IL), an aprotic solvent, and one or more unknown analytes. The processor 120 may determine the one or more voltammograms based on one or more cyclic voltammetry (CV) responses associated with the one or more unknown analytes. The one or more CV responses may include, but are not limited to, peak height, peak area, peak position, curve slope, curve shape, and/or the like. The one or more voltammograms may represent the current as a function of an applied voltage measured/detected by one or more electrodes. The X-axis of the one or more voltammograms may represent the applied voltage or the potential that is applied to the electrochemical cell. The Y-axis of the one or more voltammograms may represent the current that flows in or out of the electrochemical cell in response to the applied voltage.

The processor 120 may determine a plurality of features based on the one or more voltammograms. The plurality of features may comprise one or more shape features and one or more redox peak features. The one or more shape features may comprise one or more fitting parameters that defines the shape of the one or more voltammograms. The one or more shape features may also comprise one or more left-and-right endpoints of the one or more voltammograms. The one or more redox peak features may comprise one or more peak heights, one or more peak areas, and one or more peak potentials associated with the one or more voltammograms.

The processor 120 may determine one or more linear discriminants associated with the one or more unknown analytes based on the plurality of features. The one or more linear discriminants may be one or more data points in a linear discriminant analysis (LDA) diagram. The one or more unknown analytes (e.g., VOC mixtures) may be located in different positions in the LDA diagram. The different positions of the one or more unknown analytes in the LDA diagram may represent class separability of the one or more unknown analytes. The processor 120 may determine one or more reference linear discriminants associated with one or more known analytes. The one or more reference linear discriminants may be determined based on a machine learning model or classification model 176 described in FIG. 1B. The machine learning model or the classification model 176 may determine the one or more reference linear discriminants based on a plurality of features associated with the one or more known analytes. The one or more reference linear discriminants may be one or more reference data points. The one or more reference data points may be included in the LDA diagram. For example, the LDA diagram may comprise the one or more linear discriminants and the one or more reference linear discriminants. The one or more known analytes may be located in different positions in the LDA diagram. The different positions of the one or more known analytes in the LDA diagram may represent class separability of the one or more known analytes.

The processor 120 may classify the one or more unknown analytes based on the one or more linear discriminants and the one or more reference linear discriminants. The processor 120 may use the machine learning model or the classification model 176 to determine the one or more reference discriminants associated with the one or more known analytes. The processor 120 may determine the one or more projected means of the one or more reference linear discriminants (or the one or more reference data points) associated with the one or more known analytes. Once the one or more projected means of the one or more reference linear discriminates are determined, the processor 120 may determine one or more projected distances based on the one or more linear discriminants and the one or more projected means of the one or more reference linear discriminates. The one or more projected distances may indicate how close the one or more unknown analytes is to the one or more known analytes. The processor 120 may classify the one or more unknown analytes based on the one or more projected distances. The classification of the one or more unknown analytes may be determined based on the shortest distance among the one or more projected distances. For example, if a distance between an unknown substance and a known substance is shorter than all other distances between the unknown substance and all other known substances, the classification of the unknown substance may be determined as the known substance associated with the shortest distance.

The amplifier 130 may include an instrumentation amplifier such as a MAX4208. An amplifier 130 may be used for the sensor device 104 in order to amplify the signal received from the sensor device 104. The signals from the sensor device 104 may be very small relative to the supply voltage. The output signal may be amplified using the amplifier 130 in order to obtain optimal results from the ADC.

The memory 140 may include a volatile and/or non-volatile memory. The memory 140 may store, for example, a command or data related to at least one different constitutional element of the computing device 101. According to various exemplary embodiments, the memory 140 may store a software and/or a program 150. The program 150 may include, for example, a kernel 151, a middleware 153, an Application Programming Interface (API) 155, and/or an application program (or an “application”) 157, or the like, configured for controlling one or more functions of the computing device 101 and/or an external device. At least one part of the kernel 151, middleware 153, or API 155 may be referred to as an Operating System (OS). The memory 140 may include a computer-readable recording medium having a program recorded therein to perform the method according to various embodiments by the processor 120.

The kernel 151 may control or manage, for example, system resources (e.g., the bus 110, the processor 120, the memory 130, etc.) used to execute an operation or function implemented in other programs (e.g., the middleware 153, the API 155, or the application program 157). Further, the kernel 151 may provide an interface capable of controlling or managing the system resources by accessing individual constitutional elements of the computing device 101 in the middleware 153, the API 155, or the application program 157.

The middleware 153 may perform, for example, a mediation role so that the API 155 or the application program 157 can communicate with the kernel 151 to exchange data.

Further, the middleware 153 may handle one or more task requests received from the application program 157 according to a priority. For example, the middleware 153 may assign a priority of using the system resources (e.g., the bus 110, the processor 120, or the memory 140) of the computing device 101 to at least one of the application programs 157. For instance, the middleware 153 may process the one or more task requests according to the priority assigned to the at least one of the application programs, and thus may perform scheduling or load balancing on the one or more task requests. In an embodiment, the application program 157 may store software or methods for machine learning based VOC detection described herein.

The API 155 may include at least one interface or function (e.g., instruction), for example, for file control, window control, video processing, or character control, as an interface capable of controlling a function provided by the application program 157 in the kernel 151 or the middleware 153.

For example, the input/output interface 160 may play a role of an interface for delivering an instruction or data input from a user or a different external device(s) to the different constitutional elements of the computing device 101. Further, the input/output interface 160 may output an instruction or data received from the different constitutional element(s) of the computing device 101 to the different external device.

The display 170 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. The display 170 may display, for example, a variety of contents (e.g., text, image, video, icon, symbol, etc.) to the user. The display 170 may include a touch screen. For example, the display 170 may receive a touch, gesture, proximity, or hovering input by using a stylus pen or a part of a user's body.

The communication interface 180 may establish, for example, communication between the computing device 101 and an external device (e.g. the electronic device 102, the sensor device 104, or the server 106). In one example, the communication interface 180 may communicate with the sensor device 104 through wireless communication or wired communication. In one example, the communication interface 180 may communicate with the external device (e.g., the electronic device 102 and/or the server 106) by being connected to a network 162 through wireless communication or wired communication.

In another example, as a cellular communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. Further, the wireless communication may include, for example, a near-distance communication 164, 165. The near-distance communications 164, 165 may include, for example, at least one of Bluetooth, Wireless Fidelity (WiFi), Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication may include, for example, at least one of Universal Serial Bus (USB), High Definition Multimedia Interface (HDMI), Recommended Standard-232 (RS-232), power-line communication, Plain Old Telephone Service (POTS), and the like. The network 162 may include, for example, at least one of a telecommunications network, a computer network (e.g., LAN or WAN), the internet, and a telephone network.

In an embodiment, the computing device 101 may forward the signals or data, received from the sensor device 104, to a server 106 for processing via network 162. The server 106 may then transmit the processed data to the electronic device 102 via the network 162. In another embodiment, the computing device 101 may forward the signals or data, received from the sensor device 104, to the electronic device 102 for further processing the received signals or data.

The electronic device 102 may comprise a mobile phone, a smart phone, a tablet computer, a laptop, a desktop computer, a smartwatch, and the like. The electronic device 102 may receive sensor data of the sensors device 104 from the computing device 101 via the communication interface 180. The electronic device 102 may also receive classification data of unknown analytes from the computing device 101 via the communication interface 180. The electronic device 102 may then output the received data and/or sensor data to the user. In an embodiment, the electronic device 102 may receive the signal data from the server 106 via network 162. The electronic device 102 may receive the sensor data from the server 106 via network 162. For example, the server 106 may receive the signal and/or sensor data from the computing device 101 and perform further processing. The server 106 may then transmit the signal/sensor data or the processed signal/sensor data to the electronic device 102 to be output to the user. In an embodiment, the computing device 101 may transmit the signal data or sensor data to the electronic device 102 for further processing. In an embodiment, the electronic device 102 may include a smartphone application for interfacing with the sensor device 104 and displaying the classification data to the user.

The sensor device 104 may comprise all the components necessary to capture, process, and store electrochemical information. For example, the sensor device 104 may be used to collect measurements of current density and potential versus counter and reference electrodes in a sample. The sensor device 104 may comprise a sample that includes an ionic liquid (IL), an aprotic solvent, and one or more unknown analytes. The sensor device 104 may also comprise one or more electrodes to detect one or more electrochemical responses or one or more cyclic voltammetry (CV) responses associated with the one or more unknown analytes. It is noted that the sensor device 104 may be integrated into the computing device 101 or may be a part of the computing device 101 for VOC detection.

According to one exemplary embodiment, the server 106 may include a group of one or more servers. According to various exemplary embodiments, all or some of the operations executed by the computing device 101 may be executed in a different one or a plurality of electronic devices (e.g., the electronic device 102 or the server 106). For example, the processing of the data received from the sensor device 104 may be performed by the electronic device 102 and/or the server 106. According to one exemplary embodiment, if the computing device 101 needs to perform a certain function or service either automatically or based on a request, the computing device 101 may request at least some parts of functions related thereto alternatively or additionally to a different electronic device (e.g., the electronic device 102 or the server 106) instead of executing the function or the service autonomously. The different electronic devices (e.g., the electronic device 102 or the server 106) may execute the requested function or additional function, and may deliver a result thereof to the computing device 101 or to the electronic device 102. The computing device 101 may provide the requested function or service either directly or by additionally processing the received result. For this, for example, a cloud computing, distributed computing, or client-server computing technique may be used.

Turning now to FIG. 1B, a system 170 for machine learning based VOC detection is shown. The system 170 may be configured to use machine learning techniques to train, based on an analysis of a plurality of training datasets 172A-172B by a training module 174, and a classification model 176. Functions of the system 170 described herein may be performed, for example, by the computing device 101 and/or another computing device in communication with the computing device 101 and/or another computing device. The plurality of training datasets 172A-172B may be associated with a plurality of CV sensing data described herein. For example, the training dataset 172A may comprise one or more voltammograms associated with one or more known first substances, and the training dataset 172B may comprise one or more voltammograms associated with one or more known second substances.

The training datasets 172A, 172B may be based on, or comprise, data stored in database of the computing device 101 or the server 106 described herein. Such data may be randomly assigned to the training dataset 172A, the training dataset 172B, and/or to a testing dataset. In some implementations, assignment may not be completely random and one or more criteria may be used during the assignment, such as ensuring that various numbers of known substances and corresponding features are in each of the training and testing datasets. In general, any suitable method may be used to assign the data to the training and/or testing datasets.

The training module 174 may train the classification model 176 by forward passing the training dataset 172A and/or the training dataset 172B in a variety of ways. For example, a loss may be computed by comparing predictions to true values, using backpropagation to compute the gradient of the loss concerning each weight, adjusting the weights using the optimizer, and evaluating the model's performance on the validation set.

The classification model 176 may comprise a feature extraction module and a feature weighting module. The feature extraction module may extract features from cyclic voltammograms (CVs) by one-dimensional convolutional layers and the curve-fitting process combined with principal component analysis (PCA) or Linear discriminant analysis (LDA). The one-dimensional convolutional layers may extract shape features of the CVs as rough features and output them to the feature weighting module. The curve-fitting process utilizes several one-dimensional quadratic functions as curves to intimate (or fit) the CVs and output the parameters of the functions as detailed features to PCA and LDA. PCA and LDA may extract important features from detailed features and output them to the feature weighting module. The feature weighting module may weigh the features according to the role they provide in the discrimination process with the dense layers and yield the possibilities of VOCs in the final dense layer. In addition, the raw CV data may be processed to get more curves. For example, other curves may be obtained based on the integration of the current with respect to the voltage in the CVs. The curves generated after this processing may also be processed by the training module 174 to extract features in the same way shown above.

In an embodiment, the classification model 176 use LDA as a supervised machine learning method for dimensionality reduction, features extraction, and pattern classification. LDA may be used to remove the redundant and dependent parameters and highlight those effective parameters that contribute to efficient classification by projecting the original parameter data matrix onto a lower dimensional space, which boosts the efficiency of classification. The results of LDA may be visualized by two main dimensions, known as the first linear discriminant and the second linear discriminant, which concentrate the most significant features from the original parameters for classification. The LDA method may take into account the class labels of the data when performing the dimensionality reduction.

LDA extracted features from the training dataset 172A and/or the training dataset 172B may comprise one or more shape features and one or more redox peak features. After these LDA features are extracted, weighted, and classified, an LDA diagram may be generated. The parameters in the X-axis and the Y-axis in the LDA diagram may be the ratio of the variance, which represents the ability of the linear discriminants to discriminate different classes. Every VOC may be in different positions, and the distance between different VOCs may indicate how close their features are.

The training dataset 172A and/or the training dataset 172B may be analyzed to determine any dependencies, associations, and/or correlations between features in the training dataset 172A and/or the training dataset 172B. The identified correlations may have the form of a list of features that are associated with different labeled predictions. The term “feature,” as used herein, may refer to any characteristic of an item of data that may be used to determine whether the item of data falls within one or more specific categories or within a range. A feature selection technique may comprise one or more feature selection rules. The one or more feature selection rules may comprise a feature occurrence rule. The feature occurrence rule may comprise determining which features in the training dataset 172A occur over a threshold number of times and identifying those features that satisfy the threshold as candidate features. For example, any features that appear greater than or equal to 5 times in the training dataset 172A may be considered as candidate features. Any features appearing less than 5 times may be excluded from consideration as a feature. Other threshold numbers may be used as well.

A single feature selection rule may be applied to select features or multiple feature selection rules may be applied to select features. The feature selection rules may be applied in a cascading fashion, with the feature selection rules being applied in a specific order and applied to the results of the previous rule. For example, the feature occurrence rule may be applied to the training dataset 172A to generate a first list of features. A final list of candidate features may be analyzed according to additional feature selection techniques to determine one or more candidate feature groups (e.g., groups of features that may be used to determine a prediction). Any suitable computational technique may be used to identify the candidate feature groups using any feature selection technique such as filter, wrapper, and/or embedded methods. One or more candidate feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms used by the system 170. Instead, features may be selected on the basis of scores in various statistical tests for their correlation with the outcome variable (e.g., a prediction).

As another example, one or more candidate feature groups may be selected according to a wrapper method. A wrapper method may be configured to use a subset of features and train the classification model 176 using the subset of features. Based on the inferences that may be drawn from a previous model, features may be added and/or deleted from the subset. Wrapper methods include, for example, forward feature selection, backward feature elimination, recursive feature elimination, combinations thereof, and the like. For example, forward feature selection may be used to identify one or more candidate feature groups. Forward feature selection is an iterative method that begins with no features. In each iteration, the feature which best improves the model is added until an addition of a new variable does not improve the performance of the model. As another example, backward elimination may be used to identify one or more candidate feature groups. Backward elimination is an iterative method that begins with all features in the model. In each iteration, the least significant feature is removed until no improvement is observed on removal of features. Recursive feature elimination may be used to identify one or more candidate feature groups. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more candidate feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module 174 has generated an extracted, weighted, and/or classified feature set(s), the training module 820 may generate the classification models 178A-178N based on LDA and the feature set(s). A machine learning-based classification model (e.g., any of the classification models 178A-178N) may refer to a complex mathematical model for data classification that is generated using machine-learning techniques as described herein. In one example, a machine learning based prediction model may include a map of support vectors that represent boundary features. By way of example, boundary features may be selected from, and/or represent the highest-ranked features in, a feature set. The training module 174 may use the feature sets extracted from the training dataset 172A and/or the training dataset 172B to build the classification models 178A-1780N for each classification category (e.g., a particular substance). In some examples, the classification models 172A-172N may be combined into a single classification model 176 (e.g., an ensemble model). Similarly, the classification model 176 may represent a single classifier containing a single or a plurality of classification models 176 and/or multiple classifiers containing a single or a plurality of classification models 176 (e.g., an ensemble classifier).

The extracted features (e.g., one or more candidate features) may be combined in the classification models 178A-178N that are trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); Transformers; support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting classification model 176 may comprise a decision rule or a mapping for each candidate feature in order to assign a prediction to a class.

FIG. 1C is a flowchart illustrating an example training method 180 for generating the classification model 176 using the training module 174. The training module 174 may implement supervised, unsupervised, and/or semi-supervised (e.g., reinforcement based) learning. The method 180 illustrated in FIG. 1C is an example of a supervised learning method; variations of this example of training method may be analogously implemented to train unsupervised and/or semi-supervised machine learning models. The method 180 may be implemented by any of the devices shown in any of the systems 100 and/or 1600. At step 182, the training method 180 may determine (e.g., access, receive, retrieve, etc.) first training data and second training data (e.g., the training datasets 172A-172B). The first training data and the second training data may each comprise one or more CV sensing data or one or more voltammograms associated with one or more known substances. The training method 180 may generate, at step 184, a training dataset and a testing dataset. The training dataset and the testing dataset may be generated by randomly assigning data from the first training data and/or the second training data to either the training dataset or the testing dataset. In some implementations, the assignment of data as training or test data may not be completely random. The training method 180 may determine (e.g., extract, select, etc.), at step 186, one or more features that may be used by, for example, a classifier to differentiate among different classifications (e.g., predictions). The one or more features may comprise a set of features. As an example, the training method 180 may determine a set features from the first training data. As another example, the training method 180 may determine a set of features from the second training data.

The training method 180 may train one or more machine learning models (e.g., one or more prediction models, neural networks, deep-learning models, etc.) using the one or more features at step 188. In one example, the machine learning models may be trained using supervised learning. In another example, other machine learning techniques may be used, including unsupervised learning and semi-supervised. The machine learning models trained at step 188 may be selected based on different criteria depending on the problem to be solved and/or data available in the training dataset. For example, machine learning models may suffer from different degrees of bias. Accordingly, more than one machine learning model may be trained at 188, and then optimized, improved, and cross-validated at step 190.

The training method 180 may select one or more machine learning models to build the classification model 176 at step 192. The classification model 176 may be evaluated using the testing dataset. The classification model 176 may analyze the testing dataset and generate classification values and/or predicted values (e.g., predictions) at step 194. Classification and/or prediction values may be evaluated at step 196 to determine whether such values or classification have achieved a desired accuracy level. Performance of the classification model 176 may be evaluated in a number of ways based on a number of true positives, false positives, true negatives, and/or false negatives classifications of the plurality of data points indicated by the classification model 176. Related to these measurements are the concepts of recall and precision. Generally, recall refers to a ratio of true positives to a sum of true positives and false negatives, which quantifies a sensitivity of the classification model 176. Similarly, precision refers to a ratio of true positives a sum of true and false positives. When such a desired accuracy level is reached, the training phase ends and the classification model 176 may be output at step 198; when the desired accuracy level is not reached, however, then a subsequent iteration of the training method 180 may be performed starting at step 182 with variations such as, for example, considering a larger collection of CV sensing data or voltammograms associated with known analytes. The classification model 176 may be output at step 190.

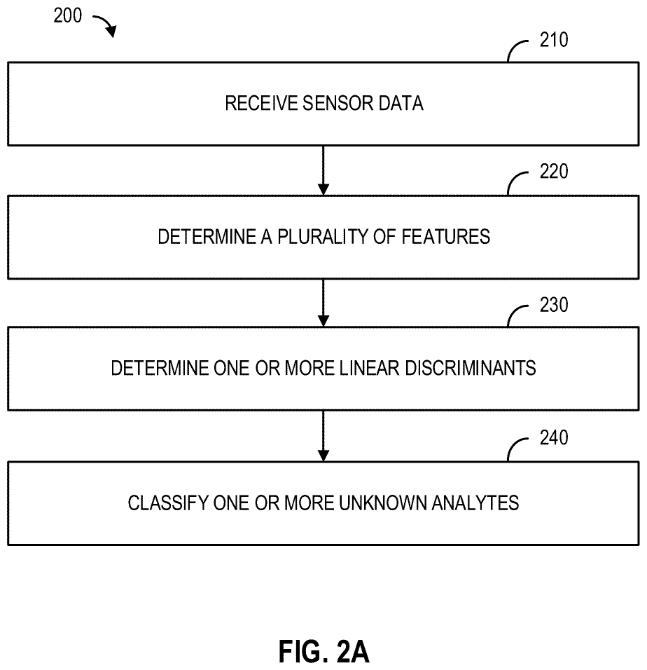

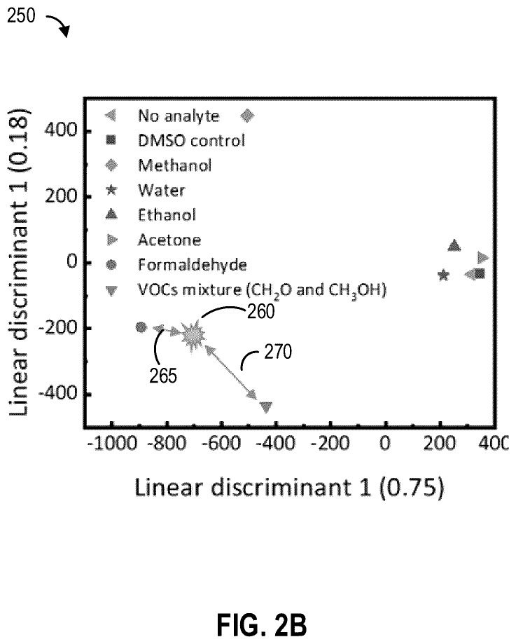

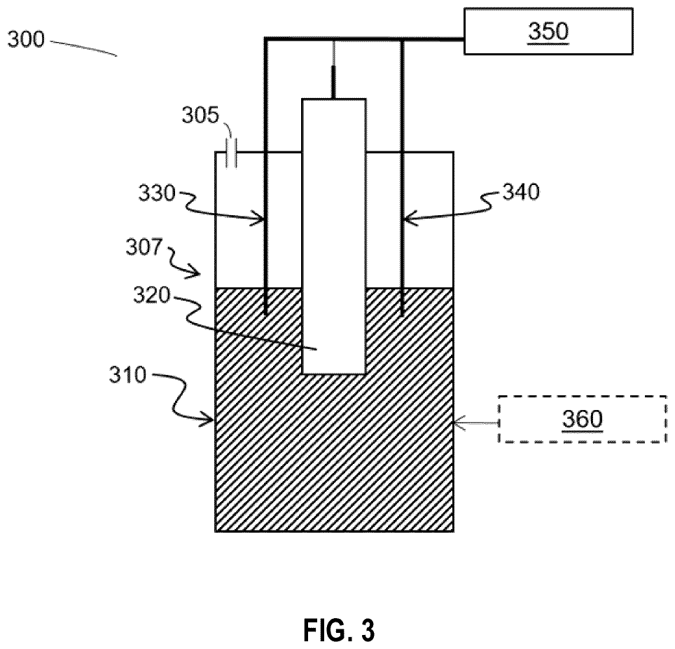

The classification model 176, once trained according the method 180, may receive one or more unknown analytes (e.g., VOC mixturea) and/or value(s) for each feature of a plurality of features associated with the one or more unknown analytes as an input(s). The classification model 176 may analyze/process the input(s) to determine a level of confidence that the one or more unknown analytes is associated with one or more particular class. For example, as shown in FIG. 2, the classification model 176 may receive one or more voltammograms for one or more known analytes and generate one or more reference discriminants for the one or more known analytes. The one or more reference discriminants may be used to determine the classification of one or more unknown analytes as described in FIG. 2.

FIG. 1D shows an exemplary process 171 of a training module for machine learning based VOC detection, which may be used in combination with any of other embodiments described herein. The process 171 of the training module (e.g., the training module 174) may comprise a convolutional layers module 177, an LDA module, a PCA module, a transformer encoder layers module, a dense layer module, and/or other machine leaning models 179. As shown in FIG. 1D, a dataset module 173 may comprise a plurality of datasets such as dataset 1 and dataset N. The feature extraction module 175 may extract or select multiple features (e.g., peak values, end points, curve fitting parameters, etc.) from the plurality of dataset. The extracted features may be provided to the LDA module 185 and the PCA module 183 for further processing. For example, a linear discriminants visualization module 181 may generate one or more LDA diagrams comprising one or more linear discriminants based on the extracted features. A comparison module 187 may compare one or more distances of the linear discriminants. The comparison module 187 may determine the shortest distance as the result. A convolutional layers module 177 may generate, based on the plurality of datasets, one or more convolutional layers. The one or more convolutional layers may be transformed one or more transformer encoder layers by a transformer encoder layers module 189. The results from the LDA module 185, the PCA module 183, and the transformer encoder layers module 189 may be combined at a dense layers module 191. The dense layers module 191 may generate one or more denser layers that are fully connected layers. The dense layers and/or output from other machine learning models 179 may be provided to a classification module 193. The classification module 193 may determine the possibility of each VOC. For example, the highest possibility of substance may be selected as the result by the classification module 193.

In an embodiment, training modules (e.g., the convolutional layers module 177, the LDA module, the PCA module, the transformer encoder layers module, the dense layer module, and/or other machine leaning models 179) for different machine learning method may be different. For example, for 1D-CNN (e.g., the convolutional layers module 177 and the dense layers module 191) or other deep learning methods (e.g., the transformer encoder layers module 189), the training modules may train a classification model (e.g., the classification model 176) by forward passing the training dataset (e.g., datasets at the dataset module 173 or the training datasets A, B 173A-B), computing the loss by comparing the predictions to the true values, using backpropagation to compute the gradient of the loss concerning each weight and adjusting the weights using the optimizer and evaluating the model's performance on the validation set. However, during the process 171, LDA may calculate class-wise means, for example, within-class scatter matrices, and/or between-class scatter matrices. Eigenvalue decomposition may then be performed to find the directions that maximize class separability. There may be no iterative optimization or backpropagation involved.

FIG. 2A shows an exemplary method 200 for machine learning based VOC detection, which may be used in combination with any of other embodiments described herein. At step 210, sensor data may be received. For example, the computing device 101 may receive sensor data. The sensor data may be received from the sensor device 104. The sensor data may indicate one or more voltammograms associated with a sample. The sample may comprise an ionic liquid (IL), an aprotic solvent, and one or more unknown analytes. The one or more unknown analytes may comprise a substance that is reactive with O₂⁻ in the ionic liquid (IL), a substance that has a different O₂ βdiffusion coefficient relative to the ionic liquid (IL), or both. The one or more voltammograms may be determined/generated based on one or more cyclic voltammetry (CV) responses associated with the one or more unknown analytes. For example, different chemicals in the one or more unknown analytes may generate different CV responses such as in peak height, peak area, peak position, curve slope, curve shape, and/or the like. The one or more voltammograms may represent the current as a function of an applied voltage measured/detected by one or more electrodes. The one or more electrodes may comprise a gold electrode as the working electrode and one or more platinum wires (e.g., two wires) as the reference and counter electrodes. X-axis of the one or more voltammograms may represent the applied voltage or the potential that is applied to the electrochemical cell. Y-axis of the one or more voltammograms may represent the current that flows in or out of the electrochemical cell in response to the applied voltage.

At step 220, a plurality of features may be determined. For example, the computing device 101 may determine the plurality of features based on/from the one or more voltammograms. Compared to voltammograms in the absence of the one or more unknown analytes, the one or more voltammograms in the presence of the one or more unknown analytes may be changed in shape and redox peaks. Thus, the one or more voltammograms in the presence of the one or more unknown analytes may be fingerprints for the one or more unknown analytes. The plurality of features may comprise one or more shape features and one or more redox peak features. The one or more shape features may comprise one or more fitting parameters. The one or more fitting parameters may be associated with the one or more voltammograms. The one or more fitting parameters may also be associated with one or more left-and-right endpoints of the one or more voltammograms. For example, a shape of a voltammogram (of the one or more voltammograms) may be represented by the equation I=aV³+bV 2²+cV+d, where I is current (A); V is potential (V); a, b, c and d are fitting parameters. The fitting parameters may be used to define the shape of different voltammograms. In addition, the one or more voltammograms may show various falls and rises at the left and right endpoints for the one or more unknown analytes. Thus, the one or more left-and-right endpoints may be determined as one of the plurality of features representing the one or more voltammograms.

The one or more redox peak features may comprise one or more peak heights associated with the one or more voltammograms, one or more peak areas associated with the one or more voltammograms, one or more peak potentials associated with the one or more voltammograms. The term “redox” may refer to reduction-oxidation involving the transfer of electrons between chemical species during a chemical reaction. Based on the one or more peak heights, the one or more peak areas, and the one or more peak potentials, kinetic activity of the one or more unknown analytes may be interpreted.

At step 230, one or more linear discriminants may be determined. For example, the computing device 101 may determine the one or more linear discriminants associated with the one or more unknown analytes based on the plurality of features. The one or more linear discriminants may be one or more data points in a linear discriminant analysis (LDA) diagram. The LDA diagram may be generated based on the plurality of features (e.g., extracted, weighted, and/or classified from the one or more voltammograms). The parameters or values representing the one or more linear discriminants in X-axis and Y-axis in the LDA diagram may indicate the ratio of variance that enables the one or more linear discriminants to discriminate different classes. The one or more unknown analytes (e.g., VOCs) may be located in different positions in the LDA diagram. The different positions of the one or more unknown analytes in the LDA diagram may represent how close their features are.

One or more reference linear discriminants may be determined. For example, the computing device 101 may determine the one or more reference linear discriminants associated with one or more known analytes. The one or more reference linear discriminants may be determined based on the machine learning model or the classification model 176 described in FIGS. 1B and 1C. The machine learning model or the classification model 176 may determine the one or more reference linear discriminants based on a plurality of features, for example, extracted, weighted, and classified based on/from the one or more known analytes. The one or more known analytes may include, but are not limited to, methanol, water, ethanol, acetone, formaldehyde, and dimethyl sulfoxide (DMSO) control. The one or more reference linear discriminants may be one or more reference data points. The one or more reference data points may be included in the LDA diagram. For example, the LDA diagram may comprise the one or more linear discriminants and the one or more reference linear discriminants. Similar to the one or more linear discriminants, the parameters or values representing the one or more reference linear discriminants in X-axis and Y-axis in the LDA diagram may indicate the ratio of variance that enables the one or more reference linear discriminants to discriminate different classes. The one or more known analytes may be located in different positions in the LDA diagram. The different positions of the one or more known analytes in the LDA diagram may represent how close their features are.

At step 240, the one or more unknown analytes may be classified. For example, the computing device 101 may classify the one or more unknown analytes based on the one or more linear discriminants and the one or more reference linear discriminants. The computing device 101 may use the machine learning model or the classification model 176 to determine the one or more reference discriminants based on the one or more known analytes. In order to classify the one or more unknown analytes, one or more projected means of the one or more reference linear discriminants may be determined. For example, the computing device 101 may determine the one or more projected means of the one or more reference linear discriminants (or the one or more reference data points) associated with the one or more known analytes. Specifically, the one or more reference linear discriminants (or the one or more reference data points) associated with the one or more known analytes may be projected to one or more vectors. A vector of known substance (of the one or more known analytes) may comprise, for example, reference linear discriminant 1 (or reference data point 1) and reference linear discriminant 2 (or reference data point 2). A vector of the mean value may be determined based on the one or more projected vectors. Similarly, the one or more linear discriminants (or the one or more data points) may be projected to one or more vectors. A vector of unknown substance (of the one or more unknown analytes) may comprise, for example, linear discriminant 1 (or data point 1) and linear discriminant 2 (or data point 2).

In an example, the projected means in LDA may be determined based on the process of transforming and/or mapping data from one or more original features (e.g., peak values, end points, curve fitting parameters, etc.) to one or more linear discriminants. This transformation and/or mappings may be achieved by using the eigenvectors derived from the matrices of the LDA process. If voltammetry is used to detect a single, known VOC analyte (or a single known substance), multiple reference points may be determined for the single known VOC analyte (or the single known substance). For example, in the LDA diagram, nine entities (or nine reference points) may be determined for the single known VOC analyte (or the single known substance). Since those nine entities (or nine reference points) are too close and/or overlapped (e.g., likely that only one entity is presented), mean of reference points may be calculated by averaging the nine or more entities for one VOC analyte (or the single known substance).

Once the one or more projected means of the one or more reference linear discriminates are determined, one or more projected distances may be determined. For example, the computing device 101 may determine the one or more projected distances based on the one or more linear discriminants and the one or more projected means of the one or more reference linear discriminates. The one or more projected distances may indicate how close the one or more unknown analytes to the one or more known analytes. In an example, a projected distance between linear discriminants (or data points) of a unknown substance (of the one or more unknown analytes) and projected means of a known substance (of the one or more known analytes) may be determined. In another example, a projected distance between a vector of a unknown substance (of the one or more unknown analytes) and a vector of projected mean of a known substance (of the one or more known analytes) may be determined. The one or more projected distance may be a Mahalanobis distance. The Mahalanobis distance may refer to a measure of the distance between a point and a distribution. It may be a generalized distance metric that accounts for correlations between variables and different variances along each dimension.

The one or more unknown analytes may be classified. For example, the computing device 101 may classify the one or more unknown analytes based on the one or more projected distances. The classification of the one or more unknown analytes may be determined based on the shortest distance determined from among the one or more projected distances. For example, a projected distance between a unknown substance and a known substance may be compared to all the projected distances between the unknown substance and all of other known substances. For example, a first distance may be between a unknown substance and formaldehyde. A second distance may be between the unknown substance and acetone. A third distance may be between the unknown substance and a VOC mixture. The computing device 101 may compare all the three distances and determine the shortest distance for the classification of the unknown substance. For example, if the first distance associated with formaldehyde has the shortest distance, the computing device 101 may classify the unknown substance to formaldehyde. In other words, after the comparison, the classification associated with the shortest projected distance may be selected for the classification of the unknown substance.

FIG. 2B shows an exemplary plot 250 of LDA results with classification determination, which may be used in combination with any of other embodiments described herein. As shown in FIG. 2B, projected distance 265 between the unknown substance 260 and a known substance, formaldehyde, may be compared with projected distance 270 between the unknown substance 260 and a known substance, VOC mixture. Since the projected distance 265 is shorter than the projected distance 270, the unknown substance 260 may be classified as formaldehyde. Alternatively or additionally, the probability of classification may be determined/provided/displayed. For example, the computing device 101 may determine/provide/display that the unknown substance 260 has 90% probability of formaldehyde and 60% probability of VOC mixture.

### Sensor Device

In various embodiments, the described method, system, and apparatus can utilize a sensor device to acquire sensing data which can then be transmitted to or received by a computing device, or incorporated into an apparatus or system. In general, the sensing device is used to collect measurements of current density and potential versus counter and reference electrodes in a sample. Referring to FIG. 3, a sensing device 300 can comprise a sample input 305 which can include any suitable mechanism (e.g., osmosis, ordinary air flow, vacuum, and the like) for collecting a sample from an environment or medium such that the sample enters a sample chamber 307.

The sample chamber 307 can comprise a medium which can include an ionic liquid and a suitable aprotic solvent, such as a polar aprotic solvent. Suitable aprotic solvents include those having low volatility, low toxicity, and high compatibility with the chosen IL. Low volatility aprotic solvents include those with relatively high boiling points, such as dimethylformamide, dimethylpropyleneurea, dimethyl sulfoxide (DMSO), hexamethylphophoramide, pyridine, and sulfolane, among others. Other suitable aprotic solvents include acetonitrile, ethyl acetate, n-methyl pyrrolidone, dimethylacetamide, and propylene carbonate. In some embodiments, the solvent in the sample chamber 307 comprises DMSO.

A variety of ILs can also be used, and the IL is not particularly limiting. In general, the IL is a room temperature molten salt at room temperature or about 25° C. which includes at least one cation and at least one anion. ILs can physically or chemically interact with different substances and exhibit different electrochemical (EC) responses, which makes them applicable for substance detection. Examples of the cations that are useful in the ionic liquid include cations of nitrogen-containing compounds, quaternary phosphonium cations, and sulfonium cations, among others.

Examples of the cations of nitrogen-containing compounds include heterocyclic aromatic amine cations, such as imidazolium cations and pyridinium cations; heterocyclic aliphatic amine cations, such as piperidinium cations, pyrrolidinium cations, pyrazolium cations, thiazolium cations, and morpholinium cations; quaternary ammonium cations; aromatic amine cations; aliphatic amine cations; and alicyclic amine cations. Examples of the imidazolium cations include 1-alkyl-3-methylimidazoliums, such as 1-ethyl-3-methylimidazolium, 1-butyl-3-methylimidazolium, 1-hexyl-3-methylimidazolium, and 1-octyl-3-methylimidazolium; 1-alkyl-2,3-dimethylimidazoliums, such as 1-ethyl-2,3-dimethylimidazolium, 1-propyl-2,3-dimethylimidazolium, 1-butyl-2,3-dimethylimidazolium, 1-pentyl-2,3-dimethylimidazolium, 1-hexyl-2,3-dimethylimidazolium, 1-heptyl-2,3-dimethylimidazolium, and 1-octyl-2,3-dimethylimidazolium; 1-cyanomethyl-3-methylimidazolium; and 1-(2-hydroxyethyl)-3-methylimidazolium. Examples of the pyridinium cations include 1-butylpyridinium, 1-hexylpyridinium, N-(3-hydroxypropyl)pyridinium, and N-hexyl-4-dimethylamino pyridinium. Examples of the piperidinium cations include 1-(methoxyethyl)-1-methylpiperidinium. Examples of the pyrrolidinium cations include 1-(2-methoxyethyl)-1-methylpyrrolidinium and N-(methoxyethyl)-1-methylpyrrolidinium. Examples of the morpholinium cations include N-(methoxyethyl)-N-methylmorpholium. Examples of the quaternary ammonium cations include N,N-diethyl-N-methyl-N-(2-methoxyethyl)ammonium and N-ethyl-N,N-dimethyl-2-methoxyethylammonium. Examples of the quaternary phosphonium cations include tetraalkyl phosphonium and tetraphenylphosphonium. Examples of the sulfonium cations include trialkylsulfonium and triphenylsulfonium.

Examples of anions that are useful in the IL include for example bis(trifluoromethylsulfonyl)imide anions ([N(SO₂CF₃)₂]—), tris(trifluoromethylsulfonyl)methide anions ([C(SO₂CF₃)₃]—), hexafluorophosphate anions ([PF₆]—), tris(pentafluoroethyl), and trifluorophosphate anions ([(C₂F₅)₃PF₃]—); boron-containing compound anions; and bis(fluorosulfonyl)imide anions ([N(FₛO₂)₂]—).

More specific examples of the IL include without limitation 1-ethyl-3-methylimidazolium bis(trifluoromethylsulfonyl)imide, 1-propyl-2,3-dimethylimidazolium bis(trifluoromethylsulfonyl)imide, 1-butyl-3-methylimidazolium bis(trifluoromethylsulfonyl)imide, 1-propyl-2,3-dimethylimidazolium tris(trifluoromethylsulfonyl)methide, N,N-diethyl-N-methyl-N-(2-methoxyethyl)ammonium bis(trifluoromethylsulfonyl)imide, 1-hexyl-3-methylimidazolium bis(trifluoromethylsulfonyl)imide, 1-octyl-3-methylimidazolium bis(trifluoromethylsulfonyl)imide, 1-ethyl-2,3-dimethylimidazolium bis(trifluoromethylsulfonyl)imide, 1-butyl-2,3-dimethylimidazolium bis(trifluoromethylsulfonyl)imide, ethyl-dimethyl-propylammonium bis(trifluoromethylsulfonyl)imide, 1-ethyl-3-methylimidazolium tris(pentafluoroethyl) trifluorophosphate, 1-hexyl-3-methylimidazolium tris(pentafluoroethyl) trifluorophosphate, 1-butyl-1-methylpyrrolidinium bis(trifluoromethylsulfonyl)imide, 1-butyl-1-methylpyrrolidinium tris(pentafluoroethyl) trifluorophosphate, methyltri-n-octylammonium bis(trifluoromethylsulfonyl)imide, 1-ethyl-3-methylimidazolium tris(trifluoromethylsulfonyl)methide, 1-butyl-3-methylimidazolium tris(trifluoromethylsulfonyl)methide, 1-hexyl-3-methylimidazolium tris(trifluoromethylsulfonyl)methide, 1-octyl-3-methylimidazolium tris(trifluoromethylsulfonyl)methide, 1-butyl-2,3-dimethylimidazolium tris(trifluoromethylsulfonyl)methide, N,N-diethyl-N-methyl-N-(2-methoxyethyl)ammonium tris(trifluoromethylsulfonyl)methide, 1-butyl-3-methylimidazolium tris(pentafluoroethyl) trifluorophosphate, 1-octyl-3-methylimidazolium tris(pentafluoroethyl) trifluorophosphate, 1-propyl-2,3-dimethylimidazolium tris(pentafluoroethyl) trifluorophosphate, 1-butyl-2,3-dimethylimidazolium tris(pentafluoroethyl) trifluorophosphate, N,N-diethyl-N-methyl-N-(2-methoxyethyl)ammonium tris(pentafluoroethyl) trifluorophosphate, 1-ethyl-3-methylimidazolium hexafluorophosphate, 1-butyl-3-methylimidazolium hexafluorophosphate, 1-hexyl-3-methylimidazolium hexafluorophosphate, 1-octyl-3-methylimidazolium hexafluorophosphate, 1-propyl-2,3-dimethylimidazolium hexafluorophosphate, 1-butyl-2,3-dimethylimidazolium hexafluorophosphate, N,N-diethyl-N-methyl-N-(2-methoxyethyl)ammonium hexafluorophosphate, 1-butylpyridinium hexafluorophosphate, 1-hexylpyridinium hexafluorophosphate, 1-cyanomethyl-3-methylimidazolium bis(trifluoromethylsulfonyl)imide, N-hexyl-4-dimethylamino pyridinium bis(trifluoromethylsulfonyl)imide, 1-(2-hydroxyethyl)-3-methylimidazolium bis(trifluoromethylsulfonyl)imide, N-(3-hydroxypropyl)pyridinium bis(trifluoromethylsulfonyl)imide, N-ethyl-N,N-dimethyl-2-methoxyethylammonium tris(pentafluoroethyl) trifluorophosphate, 1-(2-hydroxyethyl)-3-methylimidazolium tris(pentafluoroethyl) trifluorophosphate, N-(3-hydroxypropyl)pyridinium tris(pentafluoroethyl) trifluorophosphate, N-(methoxyethyl)-N-methylmorpholium tris(pentafluoroethyl) trifluorophosphate, 1-(2-methoxyethyl)-1-methyl-pyrrolidinium tris(pentafluoroethyl) trifluorophosphate, 1-(methoxyethyl)-1-methylpiperidinium tris(pentafluoroethyl) trifluorophosphate, 1-(methoxyethyl)-1-methylpiperidinium bis(trifluoromethylsulfonyl)imide, N-(methoxyethyl)-1-methylpyrrolidinium bis(trifluoromethylsulfonyl)imide, and N-(methoxyethyl)-N-methylmorpholium bis(trifluoromethylsulfonyl), and 1-butyl-1-methylpyrrolidinium bis(trifluoromethylsulfonyl)imide.

Referring again to FIG. 3, the chamber 307 will typically include a liquid phase and a gas phase for electrochemical measurements. The liquid phase 310, as described above, includes a suitable aprotic solvent and an IL. For electrochemical measurements, a working electrode 320 can be present in both the gas and the liquid phase (310) of the sample chamber 307. A variety of working electrodes can be used for this purpose as long as they have adequate conductivity. Suitable examples include gold, conductive carbon, platinum, or any other substrate with an external conductive surface. In addition to the working electrode 320, the three-electrode system also includes a counter electrode 330 and a reference electrode 340. The counter electrode 330 and the reference electrode 340 are each coupled to the working electrode 320. A variety of materials can be used for the counter and reference electrodes as is known in the art; a non-limiting example is platinum.

The three-electrode system can be coupled to an electrochemical system 350 such as a potentiostat, which can generate data which can be plotted as a voltammogram, e.g., a device for generating cyclic voltammetry data. In general, this data is typically expressed as a plot showing measurements of current density and potential versus counter and reference electrodes measured from sample chamber. Optionally, the sensor device can include an external light source 360, which can be a source capable or producing ultra-violet, visible, or near-infrared light as a means to induce increased diversity in the cyclic voltammetry sensing data and improve the ultimate classification of analytes in the sample.

### Analytes

A variety of analytes can be identified and in some instances quantified using the described method, apparatus, and system. In general, two types of analytes can be detected: first, an analyte that reacts with O₂⁻ or an analyte that has a different O₂ diffusion coefficient relative to the bare sample medium that includes the aprotic solvent and the IL, as described in more detail below. Examples include volatile organic compounds (VOCs) which can react with O₂⁻, including methanol, ethanol, formaldehyde, acetic acid, formic acid, and benzene, as well as VOCs that have a different coefficient for O₂ than the bare sample medium, including acetone, dioxane, and toluene. Moreover, a variety of other analytes can be detected, identified, and quantified, as long as they can interact with an O₂⁻ free radical. Examples include acetic acid, formate acid, and vinyl chloride. Biomolecules and viruses can also be detected. Free radicals like O₂⁻ can be indicative of pathogenic molecules in viral diseases, which means viruses can be detected. Considering that the O₂⁻ free radical is highly reactive, the detection system can detect many other substances.

### EXAMPLES

The following examples further illustrate this disclosure. The scope of the disclosure and claims is not limited by the scope of the following examples.

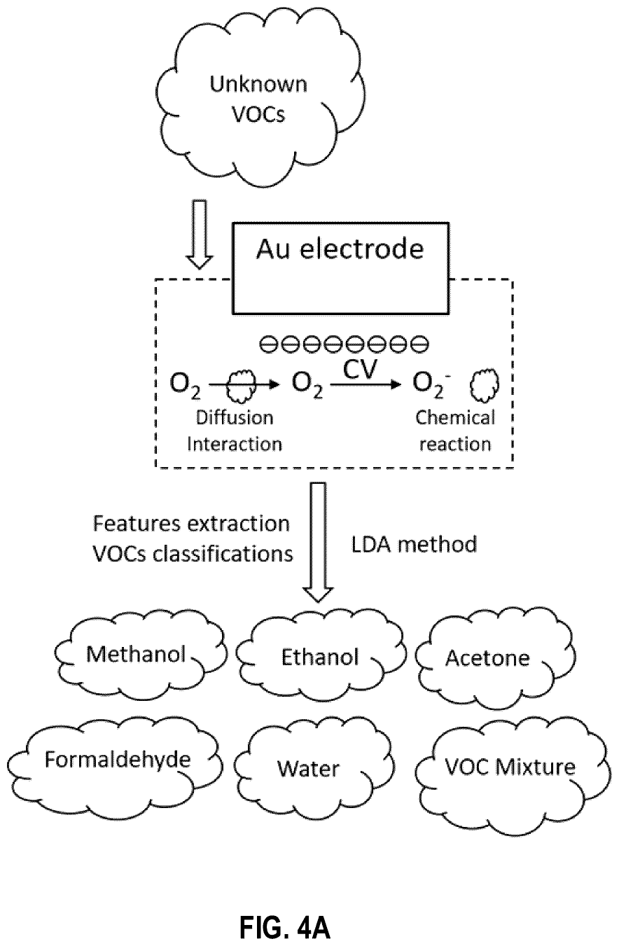

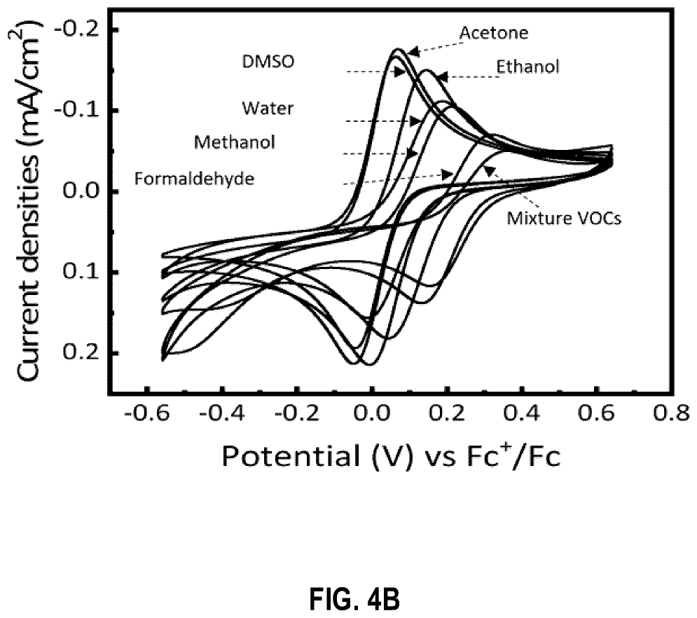

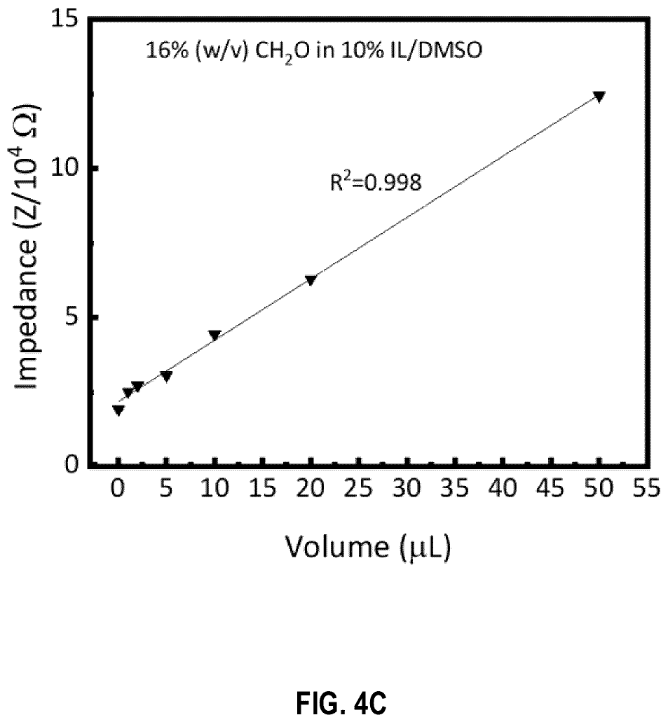

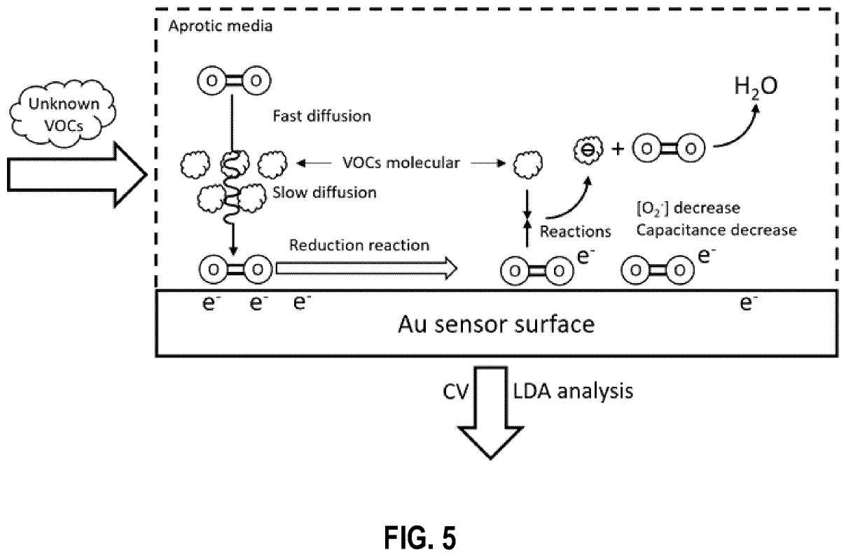

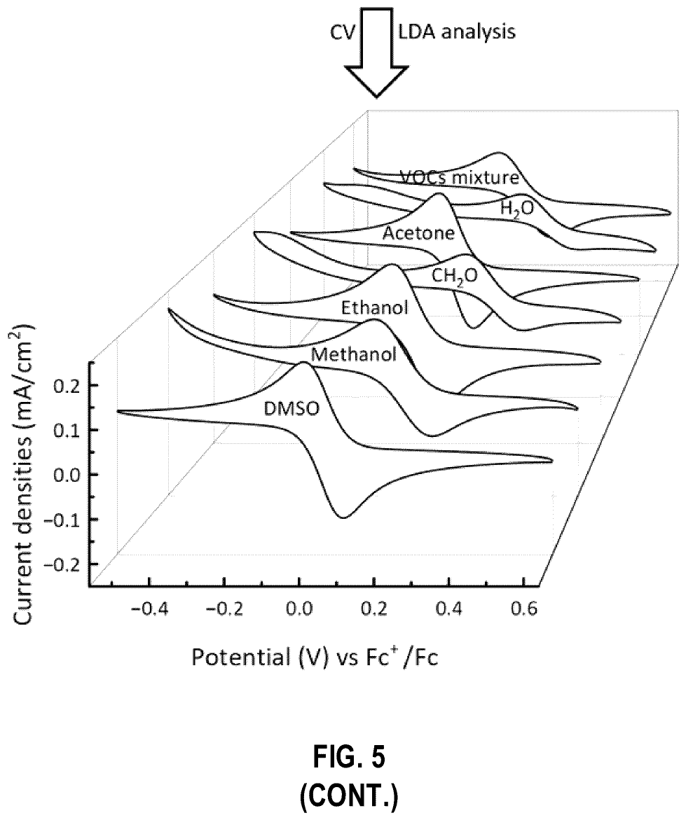

An IL-based species-selective VOC detection assay was developed using an EC three-electrode system which overcomes many limitations of existing VOC detectors. The electrolyte comprised an IL, 1-Butyl-1-methylpyrrolidinium bis(trifluoromethylsulfonyl)imide [C₄mpy] [NTf₂], and dimethyl sulfoxide (DMSO) in a specific ratio. DMSO was selected because of its low volatility, less toxicity, and high compatibility with the IL. Several types of VOCs in liquid state, including methanol, ethanol, acetone, formaldehyde, water, or their mixtures, were added into the electrolyte and evaluated by cyclic voltammetry (CV) (FIGS. 4A-C). The features of the voltammograms for each analyte were extracted and further classified by linear discriminant analysis (LDA) (see FIG. 5). The detection system showed high selectivity and could identify the VOCs as well as their mixtures. The kinetics of the interaction between VOCs and electrolyte were investigated by EC and nuclear magnetic resonance spectroscopy (NMR) techniques. The quantification of VOCs was also demonstrated using the electrochemical impedance spectroscopy (EIS) technique.

### I. Materials and Methods

### A. Materials

1-Butyl-1-methylpyrrolidinium bis(trifluoromethylsulfonyl)imide ([C₄mpy] [NTf₂], 99.5%) was purchased from IOLITEC GmbH company. Dimethyl sulfoxide (anhydrous, ≥99.9%), methanol (anhydrous, 99.8%), ethanol (200 proof, anhydrous, ≥99.5%) and acetone (ACS reagent, ≥99.5%) were purchased from Sigma-Aldrich. Formaldehyde (16% w/v in water, methanol-free) was purchased from ThermoFisher Scientific. Potassium superoxide (KO₂) and DMSO-d6 (100% Isotopic) were purchased from Fisher scientific. Deionized water (18.2 MSΩ·cm resistivity at 25° C.) was generated by Milli-Q Reference Water Purification System. ¹H and ¹³C NMR spectra were obtained on a Bruker 400 MHz NMR spectrometer at 298 K.

### B. Detection System Preparation and Measurements

To prepare IL-based electrolyte at different concentrations, 10%, 20%, 30%, 40%, 50%, 60%, 70%, 80%, 90% and 100% (v/v) of IL were mixed with DMSO. The total volume of electrolyte was 1 ml. The EC measurements were performed by CH Instruments 660e using a 2-mm gold electrode as the working electrode and two platinum wires as the reference and counter electrodes. Before any EC measurements, the gold electrode was polished by 0.05 micron Al₂O₃ powders. All three electrodes were sonicated in 0.1 M HClO₄ and DI water for 5 minutes.

To investigate the impact of IL concentrations in DMSO on its EC performance, CVs of IL/DMSO at concentrations of 10% to 100% were performed at scan rates of 36, 49, 64, 81 and 100 mV/s. To detect VOCs, the first CV measurement in the absence of analyte was performed as baseline using 10% IL/DMSO at a scan rate of 100 mV/s. Then, after adding 50 μL of VOC analyte, including methanol, ethanol, formaldehyde (diluted to 1.6% w/v water solution), acetone, water, and DMSO (as a control group) to the electrolyte and stifling for 5 minutes, the second measurement was performed as a testing result. Every CV measurement had 10 cycles. The first cycle was discarded, as it was a stabilizing process. The rest of 9 cycles were used as raw CV data and used for further analysis. The VOC determination was achieved by comparing the voltammogram of the testing result with that of the baseline.

To investigate the kinetics of species-selective detections, the interaction between VOCs and O₂⁻ were characterized by NMR spectroscopy. To prepare NMR samples, 50 uL of VOC liquids were added to 1 mg/ml of KO₂/DMSO solution. After 20 minutes, 10 uL of sample were added to 750 uL of DMSO-d6. After mixing, all samples were transferred into NMR sample tubes. Spectra were calibrated using the solvent residual peak. For methanol (HO-CH₃), ¹H NMR (400 MHz, DMSO-d6) δ 4.086 (q, J=5.2 Hz, 1H), 3.167 (d, J=4.8 Hz, 3H); ¹³C NMR (100 MHz, DMSO-d6) δ 30.68. For ethanol (HO—CH₂—CH₃), ¹H NMR (400 MHz, DMSO-d6) δ 4.341 (t, J=4.8 Hz, 1H), 3.439 (q, J=4.6 Hz, 2H), 1.054 (t, J=7 Hz, 3H); ¹³C NMR (100 MHz, DMSO-d6) δ 56.01, 18.55. For acetone (H₃C—CO—CH₃), ¹H NMR (400 MHz, DMSO-d6) δ 2.092 (s, 1H); ¹³C NMR (100 MHz, DMSO-d6) δ 31.17.

The VOCs were quantified using the following procedures: First, before adding analyte, CV was performed in IL/DMSO. Then, EIS was performed at oxidation peak potential obtained from CV measurements. This was used as a baseline for the analyte at 0 μL. Second, specific volumes (μL) of analyte were added into IL/DMSO. After stifling for 5 minutes, CV and EIS were performed to collect the data. Third, the second step was repeated multiple times to investigate the correlation between the volume of analyte and impedance of the EC system. As a result, the data for the analyte at different volumes was obtained.

### II. System Studies for IL/DMSO Electrolyte

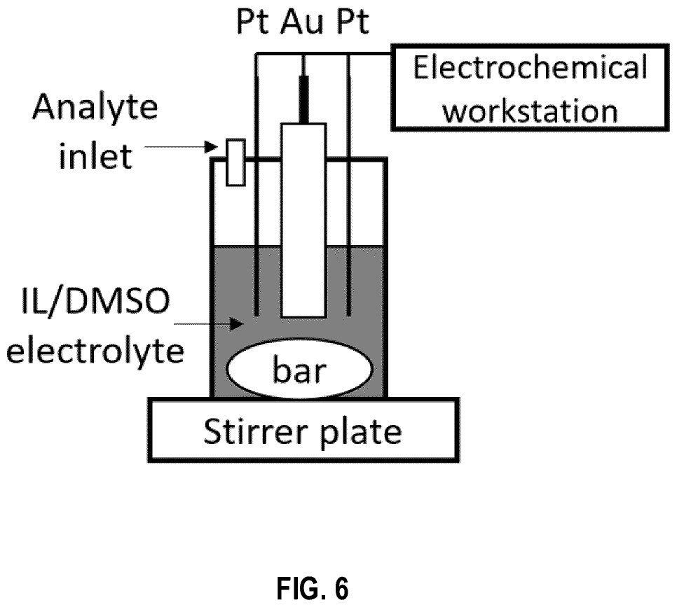

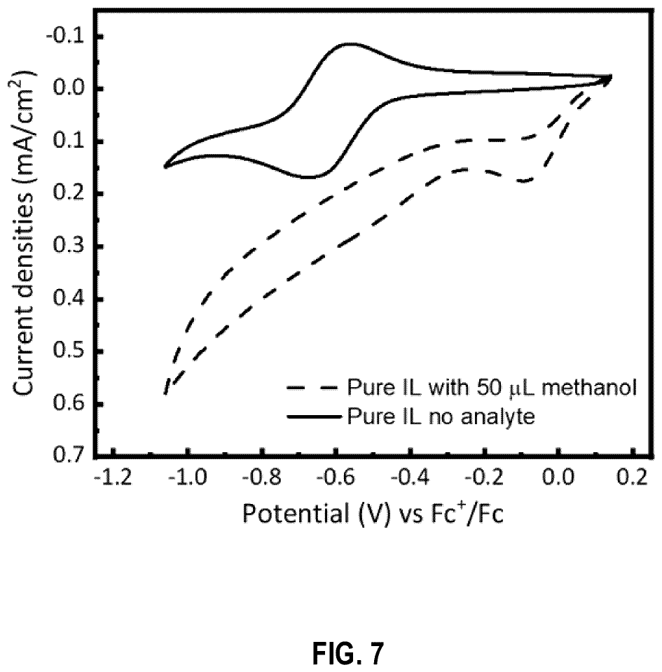

As shown in FIG. 6, the detection setup comprised a three-electrode electrochemical system with a mini stir bar. The electrodes were connected to an electrochemical workstation. The VOC analytes could be injected through the analyte inlet port. The voltammogram of pure IL is shown in FIG. 7. In the forward scan, O₂ was reduced to O₂⁻ and generated a reduction peak. In backward scan, O₂⁻ was oxidized to O₂ and generated an oxidation peak. The solid curve was the baseline as no analyte was added. When methanol (taken as the initial representative VOC compound) was added to pure IL, the shape of the voltammogram was dramatically changed (dashed curve in FIG. 7). Though the reduction peak still existed, the oxidation peak disappeared within the same potential window. This was because methanol reacted with O₂⁻ and quenched the oxidation peak. The increase of current densities during forward scanning stemmed from the reduction of water. The voltammograms of different segments were unstable and did not overlap with each other, which made the characteristics of voltammograms for each VOC analyte difficult to be defined and reproduced.

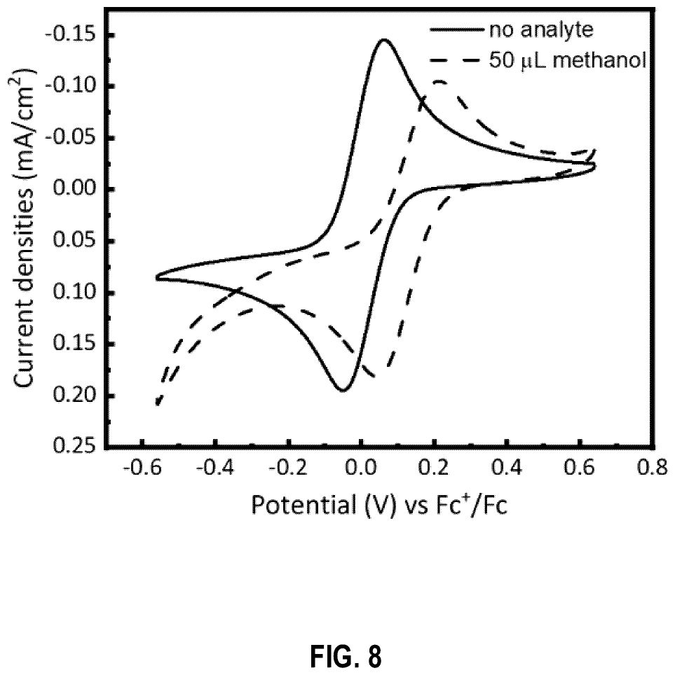

In contrast, when methanol was added to 10% IL/DMSO, the shape of the voltammogram had controllable changes (FIG. 8). Both redox peaks existed and the voltammograms of different segments were much more stable and reproducible. Thus, DMSO can improve the reversibility or reproducibility of the CV measurement. If the characteristics of voltammograms from each VOC substance is well-defined, the correlation among VOCs and their voltammograms can be established, which can be used for species-selective VOC detection.

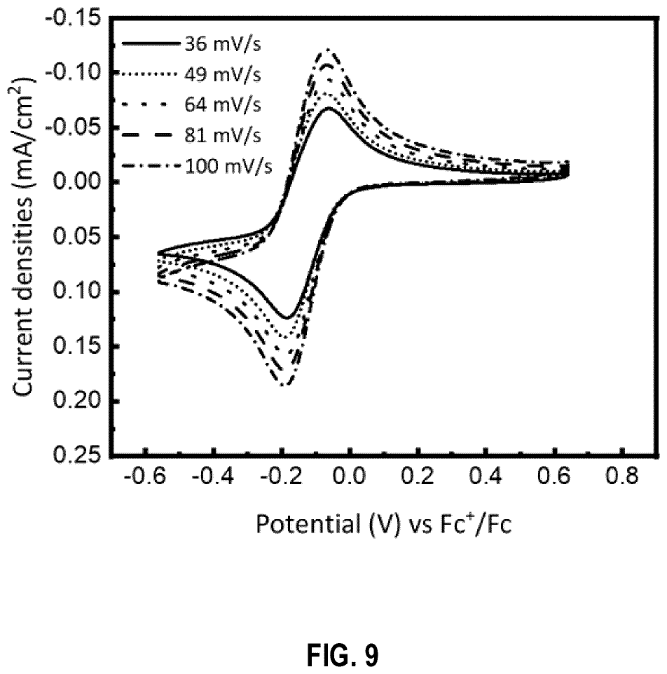

To study the impact of concentrations of IL on EC properties of IL/DMSO electrolyte, CVs of different IL/DMSO concentrations with concentrations of 10% to 100% were analyzed and provided in FIG. 9 (10%). The CVs in the IL/DMSO contained 0.01M ferrocene. It was found that the square root of scan rates was proportional to reduction peak current densities (R²>0.99), which indicated the IL/DMSO system was diffusion controlled regardless of the IL/DMSO ratio. For the reduction reaction (O₂+e=O₂⁻) during the forward scan, the reduction peak current density (jₚᵣ) was correlated to the diffusion coefficient of O₂ in the electrolyte when temperature, scan rate, and concentration of O₂ were kept constant. The larger the jₚᵣ, the faster the diffusion of O₂ to the working electrode, which was beneficial to having a faster and more accurate response for VOC analytes later. FIG. 9 was used as the voltammogram baseline in the absence of analyte. The jₚᵣ was used to calibrate the baseline of the EC detection system.

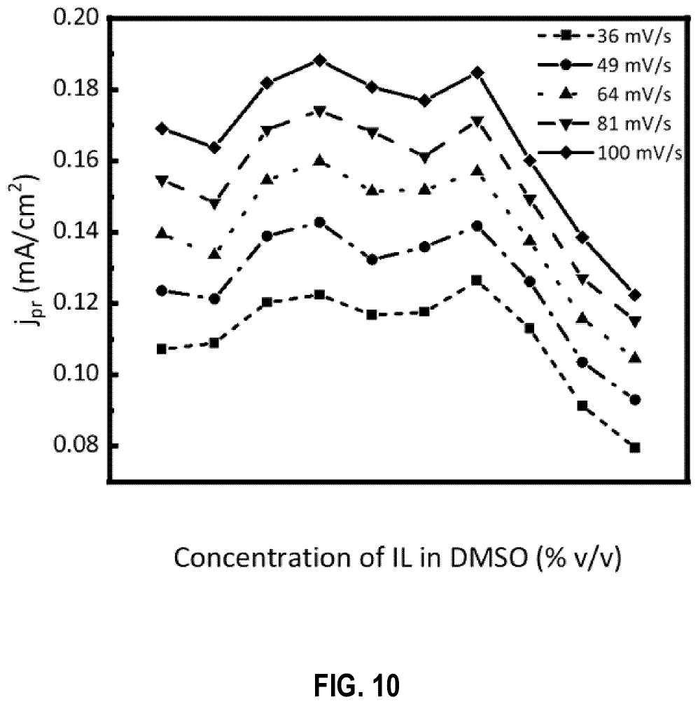

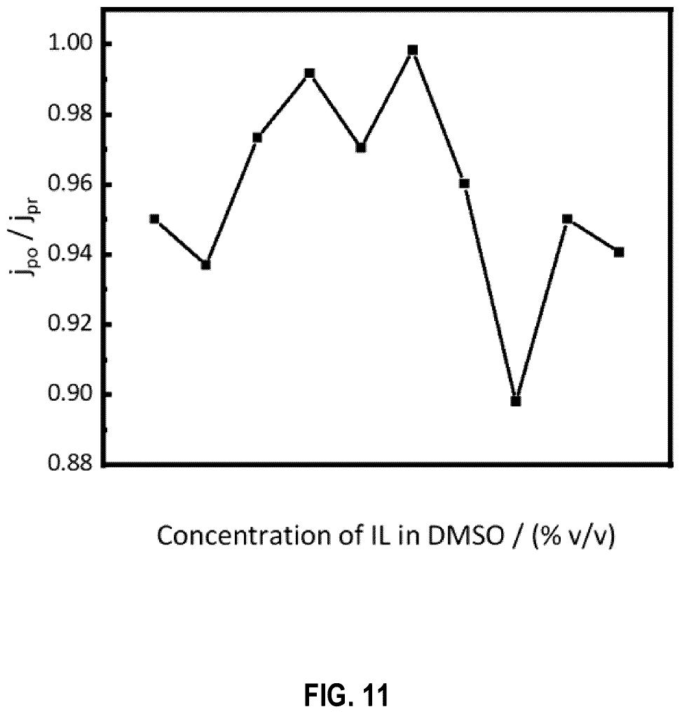

The changes of jₚᵣ with IL/DMSO in different concentrations were plotted as shown in FIG. 10. The jₚᵣ decreased after the concentration of IL was larger than 70% (see FIG. 11). The jₚᵣ is related to concentration (C) and the diffusion coefficient (D) of O₂ in electrolyte (jₚᵣ∝C·D^{0.5}), and jₚᵣ is expected to change in response to different concentration of electrolyte. O₂ has higher solubility (C) but lower diffusion coefficient (D) in IL than in DMSO, and the result of C·D^{0.5} is larger for pure DMSO than pure IL. All constants were taken from the literature. Thus, the decrease of jₚᵣ when IL %>70% is considered to be normal. The jₚᵣ was not always decreased when IL %<70%. This may be due to the interaction between the different compositions of IL and DMSO and the outcome of Cₘᵢₓ*Dₘᵢₓ^{0.5} fluctuating. The decrease of jₚᵣ may reduce the detection performance because it makes the characteristic of the voltammogram less distinguishable. To have a high detection performance, the concentration of IL could be selected from 10% to 70%. Considering the changes of CVs containing methanol in pure IL and in 10% IL/DMSO, it is possible to tune the detection resolution and range through the concentration of IL as well as the total volume of electrolyte. The ratio of jₚₒ (oxidation peak current density) to jₚᵣ is related to the reversibility of the redox reaction. When jₚₒ/jₚᵣ is equal to 1, it indicates the redox reaction is a reversible process. When analyte was added, the CV measurements can run 10 or 20 cycles as duplicate data points. Thus, the reversibility is directly correlated to the reproducibility and stability of the detection assay.

### III. VOC Classification Using Linear Discriminant Analysis (LDA)

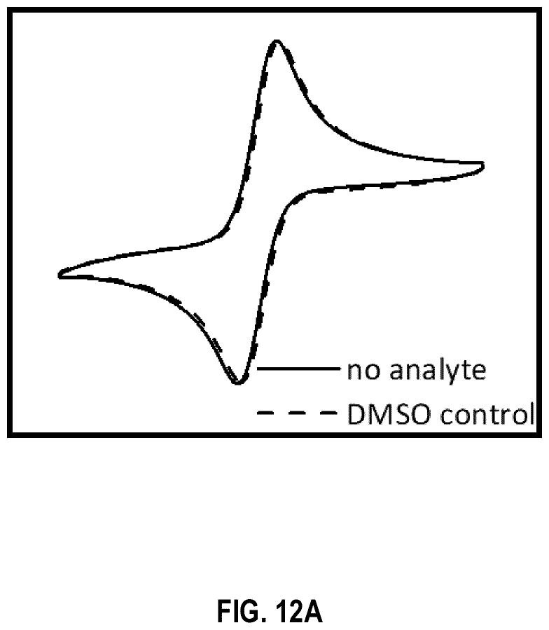

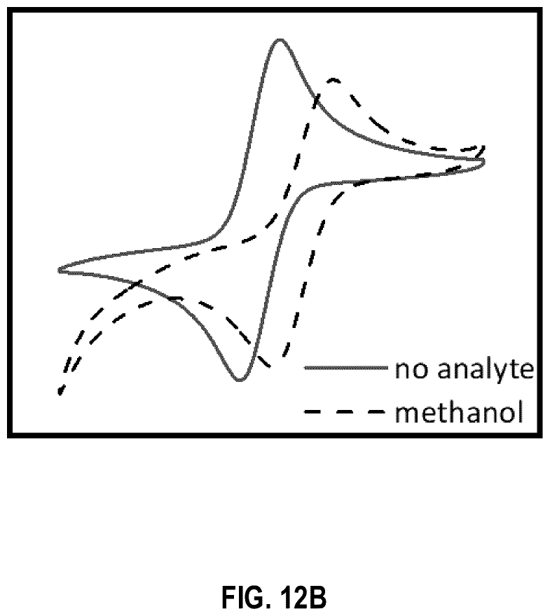

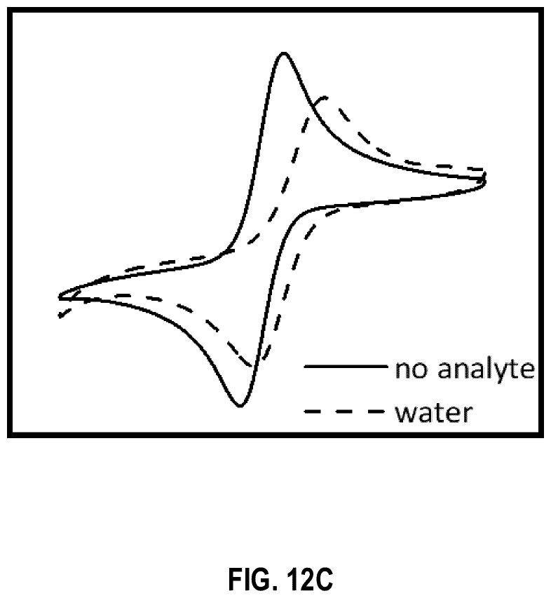

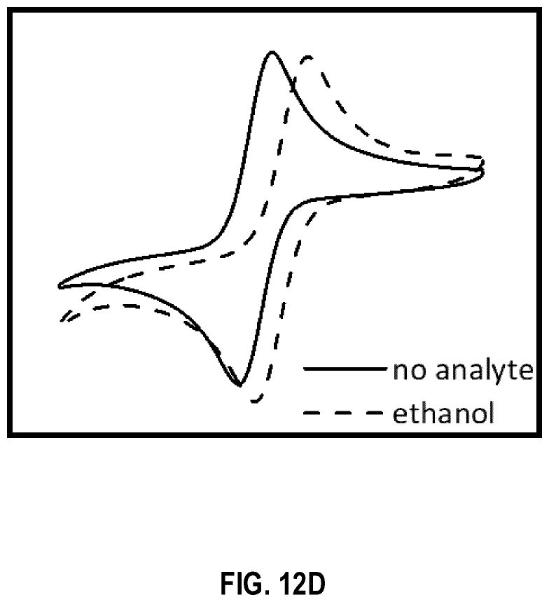

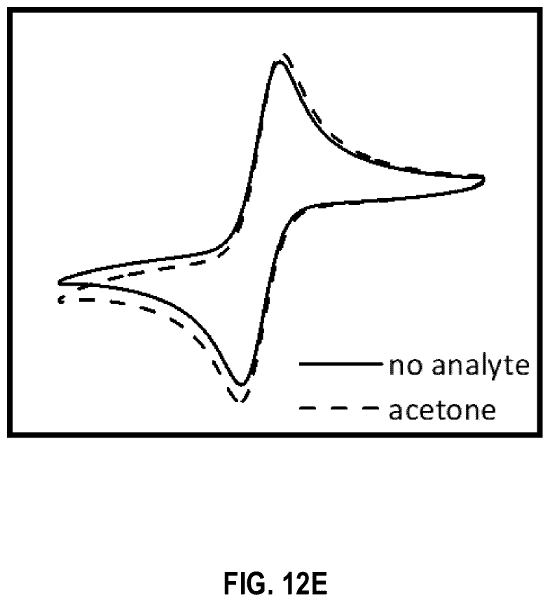

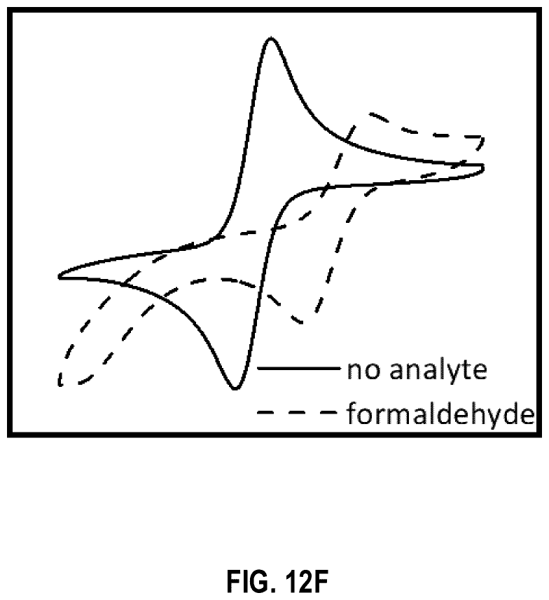

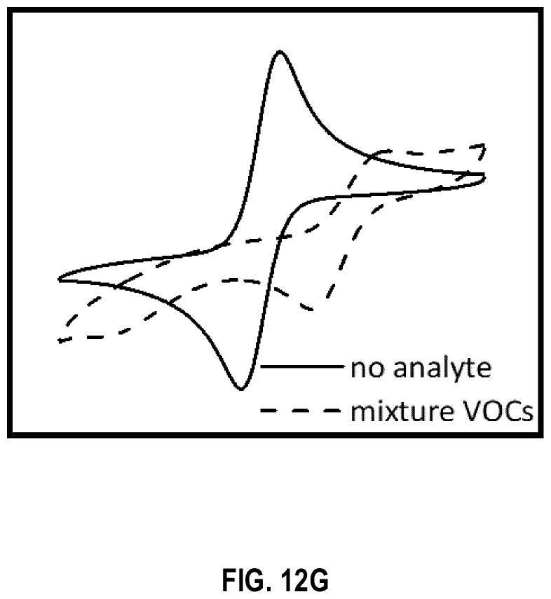

The voltammograms for the VOCs (including methanol, ethanol, formaldehyde, acetone), water, a mixture of methanol and formaldehyde, and the DMSO control are shown in FIGS. 12A-G. Before VOCs were added, the reduction peak potential was calibrated to −0.06 V (vs Fc⁺/Fc). Every VOC has its own specific characteristic voltammogram when experiment parameters were the same. Compared with voltammograms in the absence of analyte, the voltammograms in the presence of VOC analytes changed in shape and redox peaks. Thus, voltammograms were shown to be a ‘fingerprint’ for each VOC.

However, the raw CV data are in high dimensional space, which can be difficult to weigh and classify. To establish a VOC classification model, LDA was used as a supervised machine learning method for dimensionality reduction, features extraction, and pattern classification. LDA was used to remove the redundant and dependent parameters and highlight those effective parameters that contribute to efficient classification by projecting the original parameter data matrix onto a lower dimensional space, which boosted the efficiency of classification. The results of LDA can be visualized by two main dimensions, known as the first linear discriminant and the second linear discriminant, which concentrate the most significant features from the original parameters for classification. The LDA method takes into account the class labels of the data when performing the dimensionality reduction. Thus, LDA can achieve better performance in classification tasks, as it is explicitly trying to find features that separate the classes.

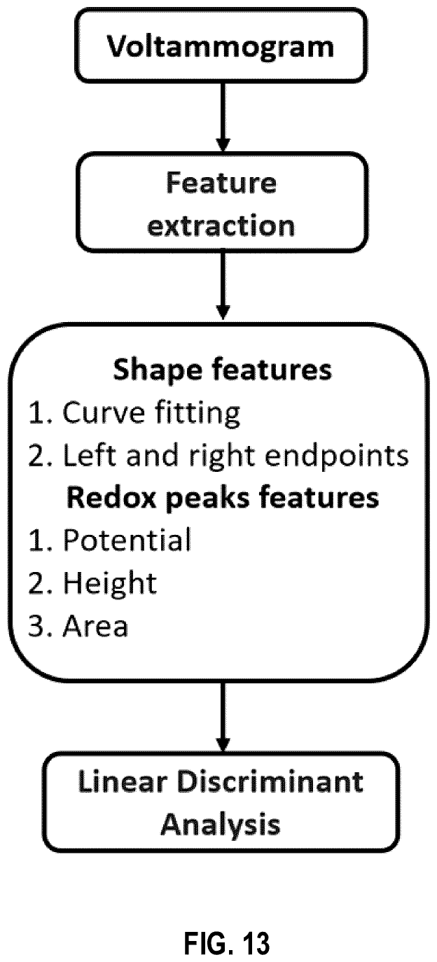

LDA extracted features of voltammograms based on their shape and redox peaks (FIG. 13). First, to define the shape of voltammograms, each CV cycle was divided into eight segments. Every segment of the total eight segments (4*2) was fitted using the following equation, individually: I=aV³+bV²+cV+d, where I is current (A); V is potential (V); a, b, c and d are fitting parameters. The shape of the voltammogram can be defined by eight equations whose fitting parameters can be used to define the shape of different voltammograms and classify VOCs. The voltammograms showed various falls and rises at the left and right endpoints for different VOCs. Thus, the left and right endpoints were specifically taken into consideration. Second, redox peaks contained information of a voltammogram, which can be used for kinetic interpretation and classification. Thus, peak height, peak area, redox peak potentials, and difference between redox peak potentials were also considered as parameters.

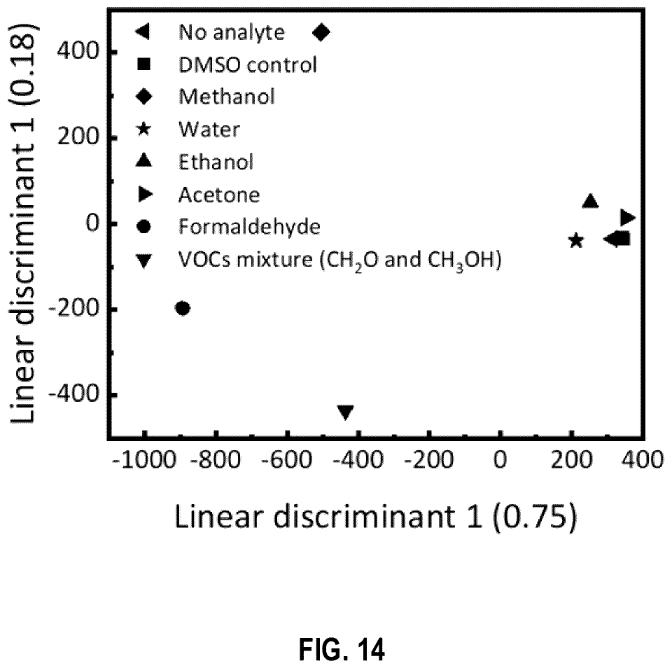

After these features were extracted, weighted, and classified, an LDA diagram was generated as shown in FIG. 14. To maximize class separability, LDA increases the inter-class variance while decreasing the intra-class variance. Every VOC contains 7-9 datapoints from 10 cycles of CV measurements in the LDA diagram, and they overlap as their features are the same. The parameters in the X and the Y axis are the ratio of the variance, which represents the ability of the linear discriminants to discriminate different classes. Every VOC was in different positions, and the distance between different VOCs represented how close to their features they were.

Due to 10% IL/DMSO being used, when 50 μL of DMSO were added to the electrolyte, the voltammogram was supposed to show no change, which aligned well with experiment results (FIG. 12A). From the LDA diagram, ‘No analyte’ and ‘DMSO control’ are very close and almost overlap with each other, which means their voltammogram features are highly identical. Thus, ‘No analyte’ and ‘DMSO control’ can be used as control groups. The features of ‘Ethanol’, ‘Acetone’ and ‘Water’ are close to the control group but different in detail. Such differences would have been difficult to be weighted and quantified manually but now are recognized as different categories by the LDA model.

The voltammograms for ‘Methanol’ and ‘Formaldehyde’ have greater changes compared with other VOCs (FIGS. 12B and 12F). Their positions are far away from the others. The system can also identify the mixture of ‘Methanol’ and ‘Formaldehyde.’ The voltammogram for the VOCs mixture was different from pure voltammograms. Each VOCs mixture was separated from others, even when the mixture containing acetone which is a type of VOC that made relatively smaller difference in its voltammogram (FIG. 12E) than other VOCs. From the changes of voltammograms, impacts from different species overlap, and both contribute to the changes of voltammograms. For example, the oxidation peak for VOCs mixtures did not significantly drop, compared with pure methanol and ethanol (FIGS. 12B and 12D), because the oxidation peak was compensated by the presence of acetone which led the oxidation peak increase (FIG. 12E). When the classification model was established, new unknown CV data can be input into the model for classification.

To test the reliability of the classification system, acetone was tested in 10% IL/DMSO electrolyte that contained 1 wt % of TiO₂ nanoparticles. TiO₂ nanoparticles are sensitive to acetone and have been used to fabricate acetone sensors and other types of energy devices. However, the presence of TiO₂ nanoparticles did not affect the detection of acetone; the voltammogram for acetone in the presence of TiO₂ nanoparticles was identical to the case in the absence of TiO₂ nanoparticles (FIG. 12E). This result confirmed the classification system was able to precisely identify VOCs without interference by solid impurities, even if they are sensitive to the analyte.

The classification system was also able to test VOCs at different temperatures. Voltammograms for methanol at 25, 35 and 45° C. were analyzed. Although their voltammograms were different, the shape of the voltammograms did not change. The redox peak current was increased, as the increase of temperature increased the diffusion coefficient of reactant. Superior to traditional commercial sensors that detect and evaluate a single variable, the LDA model can classify each voltammogram based on conclusive information and ignore local differences. Reproducibility of the detection system was also tested. The results of triplicate CV measurements for three Methanol samples were analyzed. The features from three voltammograms were highly identical to each other, which exhibits excellent reproducibility of the CV measurements. The data from the triplicate tests was further analyzed for LDA classification, and the Methanol data from three different tests could still be categorized to one VOC type, as they overlap with each other. This confirms the high producibility of the sensor data as well as the classification result.

For VOC detection, the resolutions were different for different VOCs depending on how fast the reaction between each VOC and O₂⁻ was, which could be seen from the change of voltammograms. To best identify as many types of VOCs as possible, the volume of all VOCs was kept at 50 μL. After converting volume to molarity, the resolution for the VOCs classification system was 1.240 mmol for methanol, 0.858 mmol for ethanol, 0.267 mmol for formaldehyde (1.6 wt %), 0.676 mmol for acetone, and 2.780 mmol for water. Thus, the average resolution for different VOCs was around 1.164 mmol. The resolution can further be optimized by adjusting the composition and volume of the electrolyte. Considering the potential window was 1.2 V and the scan rate was 0.1 V/s, the response time for each measurement can be as fast as 24 seconds, which is comparable with most of existing sensor technologies.

The chemical reactions behind the species-selective detection are explained as follows based on VOC voltammograms and NMR spectra, without wishing to be bound by any theory. When the reduction reaction occurred in the CV process, O₂⁻ was produced and interacted with VOCs, which caused the shape of voltammograms to change. The shape of voltammograms for the three VOCs shared similar features: 1) both redox peaks shifted to more positive potential; 2) height of oxidation peak decreased, which was evidence of the consumption of O₂⁻ by AH, as the height of oxidation peak is proportional to the concentration of O₂⁻; 3) fall and rise at left and right endpoints in voltammograms. The extent of the shape changes from the largest to the smallest have relationship as: methanol>water>ethanol. This aligns well with the rate constants for reactions between O₂⁻ and the three VOCs where methanol (k₂=1.1*10⁷ M⁻¹s⁻¹)>water (k₂=1.0*10⁵M⁻¹s⁻¹)>ethanol (k₂=1.42*10² M⁻¹s⁻¹).

Without being bound by any theory, the chemical reaction between formaldehyde solution and O₂⁻ is believed to proceed according to the following equation: O₂⁻+CH₂O→H₂O+CO₂↑. Due to the presence of H₂O in the formaldehyde solution, O₂⁻ reacted with H₂O and produced a hydroxyl radical. The hydroxyl radical reacted rapidly with formaldehyde to produce H₂O and CO₂. Both H₂O and CO₂ can react with O₂⁻ and lead the shape of voltammogram to change dramatically. This is likely the reason why the shape of voltammogram for formaldehyde changed the most. Acetone does not directly react with O₂⁻, which aligned well with its CV result where only the redox peak current increased slightly (FIG. 12E). Based on principles of electrochemistry, the peak current is associated with the concentration of reactant (O₂), scan rate, and diffusion coefficient of reactant (O₂) in the electrolyte. The only changeable parameter is the diffusion coefficient. O₂ has a slightly larger diffusion coefficient in acetone (6.68*10⁻⁹ m²/s) than in DMSO (10⁻⁹ m²/s). After adding acetone, the diffusion coefficient of O₂ in the electrolyte mixture has also been changed, and resulted in different voltammograms. FIG. 12E shows the voltammogram for the electrolyte containing acetone that has larger redox peaks than the voltammogram for pristine electrolyte, which aligns with their changed diffusion coefficients.

To further confirm the reactions between VOCs and O₂⁻, solutions containing one of four VOCs (methanol, ethanol, acetone, or formaldehyde) and 1-fold dilution or 10-fold dilution KO₂ were analyzed by ¹H and ¹³C NMR. Literature chemical shifts were used for methanol, ethanol, acetone, and water. As expected, NMR revealed no peak shifts for KO₂ solutions containing methanol and ethanol. This is consistent with the kinetics of the expected reaction. For acetone, the chemical shift for H in the methyl groups was at 2.09 ppm; the chemical shift for C in the methyl group was at 31.17 ppm in the ¹³C NMR spectrum; the chemical shift for the carbonyl carbon is normally at 206.31 ppm but was missing due to low volume (0.5 uL). Acetone was not expected to react with O₂⁻, which was consistent with the NMR results. For formaldehyde (CH₂O), no peaks were detected after it reacted with KO₂, indicating that all CH₂O was completely consumed. The reaction between CH₂O and KO₂ was rapid. A large number of bubbles were generated during the reaction, which is believed to be CO₂ gas as described above.

The water product was further quantified to confirm the expected chemical reactions. It is challenging to completely remove the residual water in the DMSO-d6 solvent. Therefore, the concentrations of the residual water from the different samples that were not derived from the chemical reaction were assumed to the same because all DMSO-d6 was taken from the same bottle, and all NMR tubes were used following the same drying procedures. All the reactions time were controlled to be the same.

To qualitatively determine the generation of H₂O in the expected reactions, 1-fold and 10-fold dilution of a KO₂ solution was added to 1 ml of DMSO that contained 50 uL of different VOCs. The presence of H₂O generated a peak at 3.33 ppm in ¹H-NMR spectrum. As the rate constants for the reactions between O₂⁻ and the three VOCs are given the order of methanol (k₂=1.1*10⁷ M⁻¹s⁻¹)>water (k₂=1.0*10⁵ M⁻¹s⁻¹)>ethanol (k₂=1.42*10² M⁻¹s⁻¹). The reaction rates of each VOCs and water are also expected to be in the order of methanol>water>ethanol. The amount of water produced from the reactions should also be in the order of methanol>ethanol. The ratio of the integrals from the 1-fold and 10-fold dilution sample can be used to determine the change of H₂O content.

Based on the H₂O peak (3.33 ppm) area ratio of VOCs in 1-fold KO₂ solution to 10-fold dilution KO₂ solution, it was found that H₂O content was much higher in 1-fold samples than in 10-fold samples for methanol, ethanol, and CH₂O. The 10-fold dilution sample contained much less O₂⁻ reactant. The water product from methanol was much higher than that from ethanol, which confirmed the anticipated chemical reactions. Acetone should not react with O₂⁻ and its H₂O content showed negligible changes, which can be used as a baseline control for the other three VOCs. These results were consistent with the above discussion regarding the kinetics of the species-selective detection for VOCs.

Based on these results, the system can detect two types of VOCs, either a VOC that can fast react with O₂⁻ or a VOC that has a different diffusion coefficient for O₂ relative to the pristine electrolyte. Common VOCs that can react with O₂⁻, including methanol, ethanol, formaldehyde, acetic acid, formic acid and benzene, and VOCs that have a different coefficient for O₂ than the electrolyte, including acetone, dioxane and toluene, can easily be detected by the system.

Moreover, the detector can differentiate other chemicals, as long as they can interact with the O₂⁻ free radical, such as acetic acid, formate acid and vinyl chloride. Biomolecular and viruses can also be detected by the system. Free radicals like O₂⁻ could be pathogenic molecules in viral disease pathogenesis, which means some viruses are also detectable by the detection system. Considering the O₂⁻ free radical is highly reactive, the detection system can detect many other substances.

### IV. VOCs Quantification and Monitoring

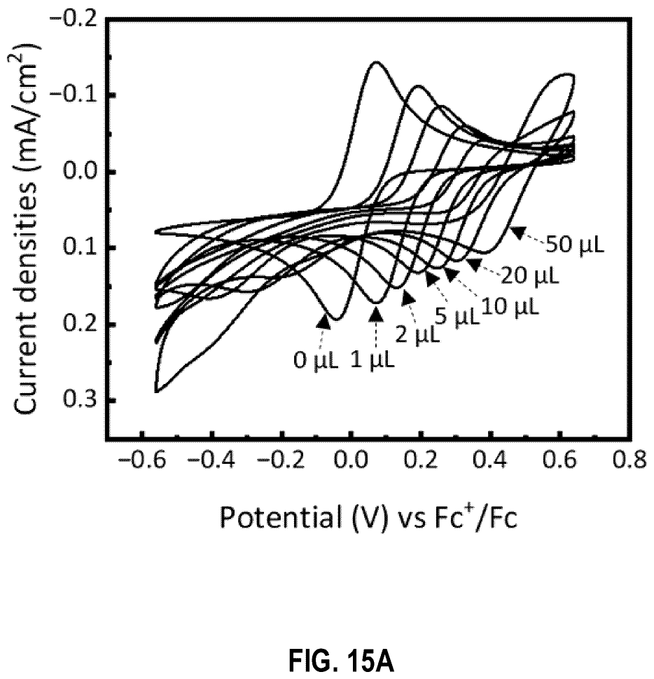

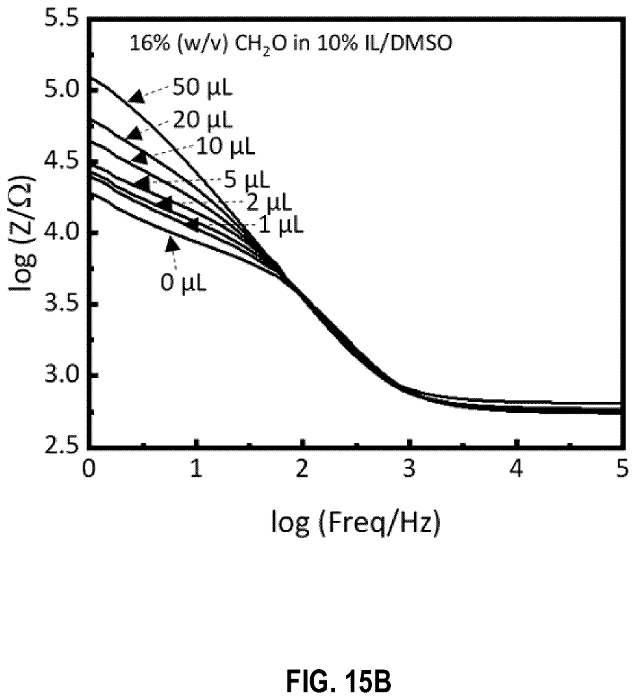

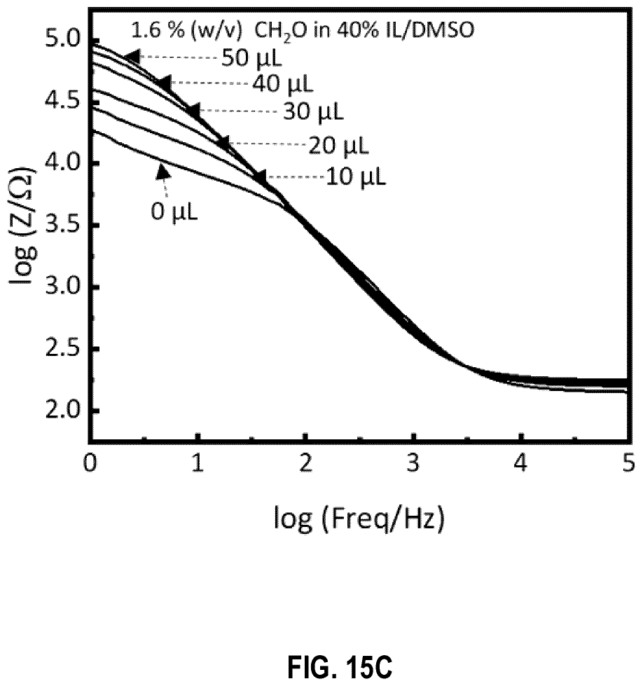

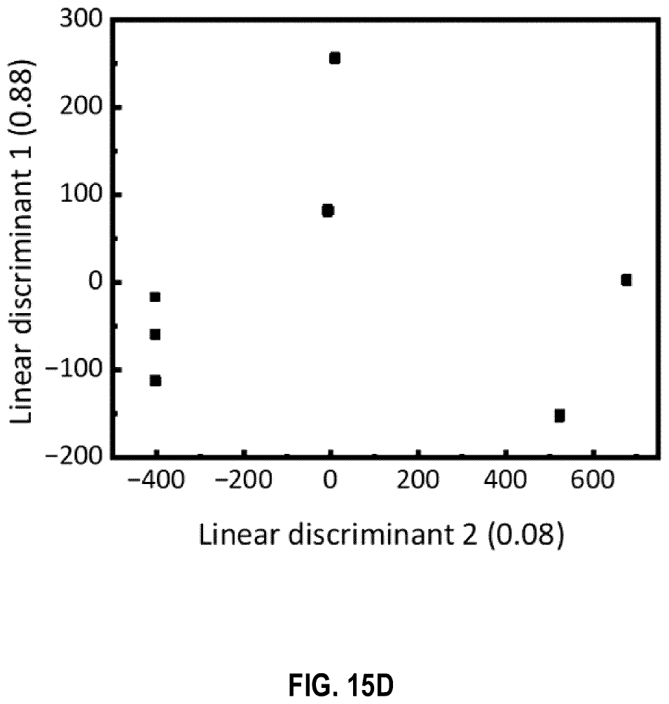

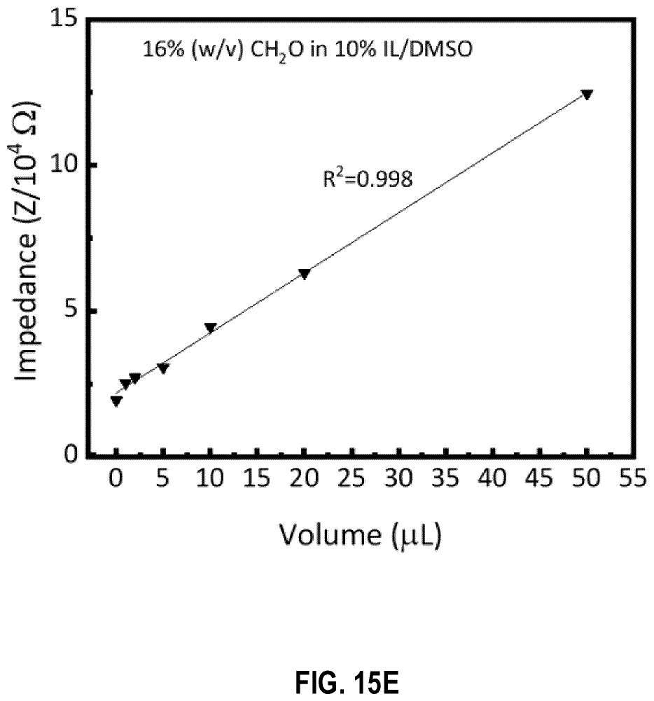

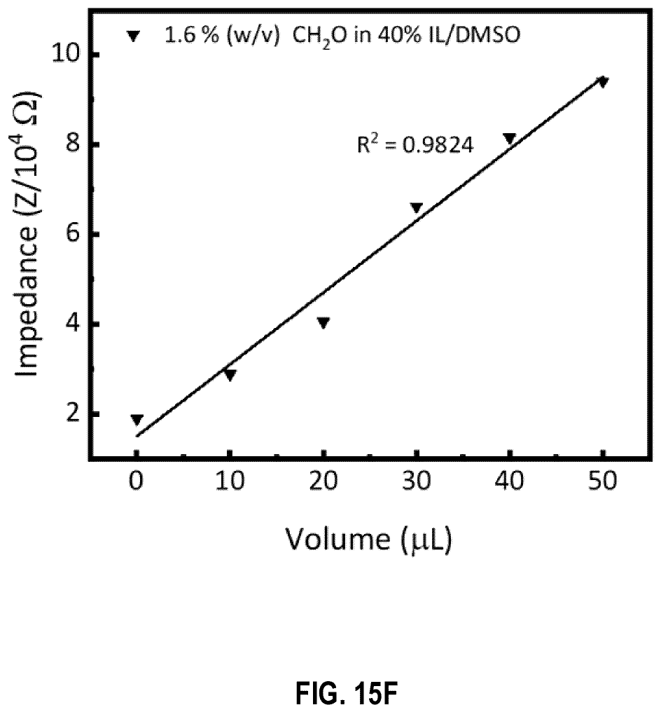

In addition to using LDA method to classify different kinds of VOCs, LDA can also be used for VOCs quantification. CH₂O was used as a representative VOC to demonstrate the quantification system. As it shown in FIG. 15A, the CVs in the 10% IL/DMSO electrolyte changed with increasing volume of CH₂O. The H₂O that was produced from the reaction of O₂⁻ and CH₂O further interacted with O₂⁻, which resulted in a positive shift of the voltammograms and the decrease of height of redox peaks. This change of the voltammograms can be analyzed by the LDA method. The LDA can analyze the features of voltammograms containing different volumes of CH₂O. Once analyte containing unknown volume of CH₂O is added to the electrolyte, the LDA can categorize it to specific volume from its voltammogram curve.

As shown in FIG. 15D, different amounts of CH₂O are clearly categorized to different groups, and each group is separated from others. With the increase of CH₂O volume, the X and Y of the LDA data points tend to become smaller. Such change tendencies align well with the change of voltammograms (FIG. 15A), where the CV curves shift to the right when CH₂O volume increased. As a result, the limit of detection is at least 1 μL. The detection range was 0 to 50 μL.

Other than using LDA, a traditional electrochemical method that combines the CV and EIS techniques can be used to quantify the CH₂O volume without a classification function. For each unknown analyte, CV was performed first, and then EIS was performed at oxidation peak potential. The EIS spectra for 10% IL/DMSO system containing different volumes of 16% (w/v) CH₂O solution are shown in FIG. 15B. The impedance at low frequencies increased with the increase of CH₂O level. The impedance at 1 Hz and volume level of CH₂O was also plotted. Their linear correlation (R²=0.998) is shown in FIG. 15E. With this correlation, the CH₂O level was able to be predicted from the impedance of the IL/DMSO electrochemical system. The quantification from LDA can be better than that from the electrochemical method because the data points that represent different volumes of CH₂O are farther away in the LDA method (FIG. 15D), while the 1-μL point is very close to 2-μL point in electrochemical method (FIG. 15E). This suggests that the LDA method can analyze comprehensive features from the voltammogram and differentiate smaller interferences from the analyte source than the EIS, which only takes a single parameter, impedance, into consideration. However, this system could be coupled with the LDA method as an alternative to monitor VOC concentration while simultaneously classifying different types of VOCs in real-time.

The detection range can be tuned by adjusting the concentration of IL. For example, 10% IL/DMSO was not able to quantify the 1.6% (w/v) CH₂O solution, as the concentration of CH₂O was too low for the impedance to show any differences. However, 40% IL/DMSO worked for quantification of the 1.6% (w/v) CH₂O solution. The corresponding EIS spectrum and the linear correlation (R²=0.9824) are shown in FIGS. 15C and 15F. Considering the CV was performed ahead and could be sent for LDA analysis, the quantification was also a species-selective measurement. These results demonstrated the species-selective quantification for VOCs with tunable detection range for a 1.6% and a 16% (w/v) CH₂O solution. The limit of detection was as low as 1 μL. After converting volume to moles, the linearity calibration range was determined to be from 5.30 μmol to 53.00 μmol with a limit of detection at 0.53 μmol.

The kinetics of the quantification is associated with capacitance. In EIS, the relationship between impedance and capacitance is given by:

$$Z ( ω ) = \frac{1}{j ω \times C ( ω )}$$

where ω is phase, and Z(ω) and C(ω) are impedance or capacitance at phase ω. During the detection, when the CH₂O level increased, Z also increased at low frequencies (FIGS. 15e and 15F). The increase of Z resulted in decrease of C. C is closely related to the surface charge of the Au working electrode. During the redox reaction, O₂⁻ was produced and diffused on the surface of the working electrode, which was the main contributor for the surface charge. When O₂⁻ interacted with CH₂O, CH₂O was quickly consumed by O₂⁻. As a result, surface charge of the Au electrode became lower, causing a decrease of C.

The sensitivities of the detection system for the VOCs species were different, as each VOC has different properties. Considering the detection system using characteristic voltammograms to classify VOCs, any volume of VOC analyte that results in a minimal and distinguishable change of voltammograms is considered to be the minimum detection volume. CVs for 10% IL/DMSO containing different volumes of four VOCs species were analyzed to further study detection volume. The minimum volumes that result in a distinguishable different voltammogram are 5 μL for methanol, 10 μL for ethanol, 1 μL for formaldehyde, and 50 μL for acetone. Methanol, ethanol, and formaldehyde were detected by the chemical reaction with O₂⁻ that generated from the redox reaction during the CV process. Methanol has a larger rate constant when reacting with O₂⁻ than ethanol. Thus, methanol has a higher sensitivity than ethanol. Formaldehyde has the highest sensitivity because formaldehyde can fast react with O₂⁻. After the reaction, the formaldehyde was completely consumed by O₂⁻ based on the ¹³C NMR results. Acetone has the lowest sensitivity, because it was detected by the change of diffusion coefficient of O₂ instead of the chemical reaction. As a result, the minimum amounts of VOCs for the detection in a 1-ml scale of electrolyte are 0.124 mmol for methanol, 0.172 mmol for ethanol, 0.005 mmol for formaldehyde, and 0.676 mmol for acetone in a 1 ml of 10% IL/DMSO detector. It will be understood that the limit of detection of the system can vary widely depending on various parameters. The appended claims are therefore not limited to any particular detection limit.

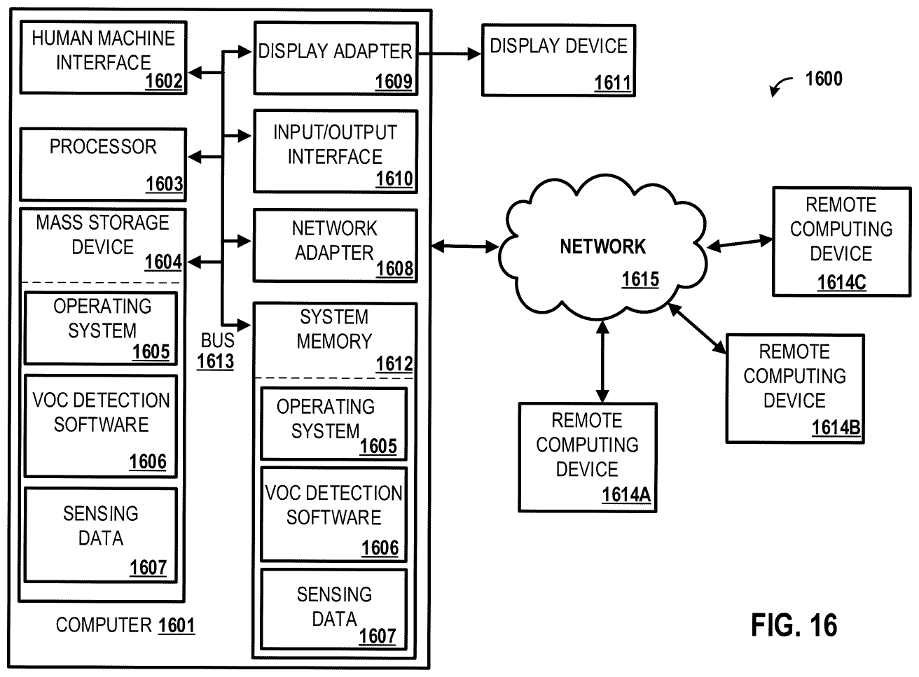

In an example, the methods and systems may be implemented on a computer 1601 as shown in FIG. 16 and described below. By way of example, any of the devices shown in any of the systems 100 may be a computer 1601 as shown in FIG. 16. Similarly, the methods and systems disclosed may utilize one or more computers to perform one or more functions in one or more locations. FIG. 16 is a block diagram illustrating an exemplary operating environment 1600 for performing the disclosed methods. This exemplary operating environment 1600 is only an example of an operating environment and is not intended to suggest any limitation as to the scope of use or functionality of operating environment architecture. Neither should the operating environment 1600 be interpreted as having any dependency or requirement relating to any one or combination of components illustrated in the exemplary operating environment 1600.

The present methods and systems may be operational with numerous other general purpose or special purpose computing system environments or configurations. Examples of well-known computing systems, environments, and/or configurations that may be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems may be performed by software components. The disclosed systems and methods may be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods may also be practiced in grid-based and distributed computing environments where tasks are performed by remote processing devices that are linked through a communications network. In a distributed computing environment, program modules may be located in local and/or remote computer storage media such as memory storage devices.

Further, one skilled in the art will appreciate that the systems and methods disclosed herein may be implemented via a general-purpose computing device in the form of a computer 1601. The computer 1601 may comprise one or more components, such as one or more processors 1603, a system memory 1612, and a bus 1613 that couples various components of the computer 1601 comprising the one or more processors 1603 to the system memory 1612. In the case of multiple processors 1603, the computer 1601 may utilize parallel computing.

The bus 1613 may comprise one or more of several possible types of bus structures, such as a memory bus, memory controller, a peripheral bus, an accelerated graphics port, or local bus using any of a variety of bus architectures. By way of example, such architectures may comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 1613, and all buses specified in this description may also be implemented over a wired or wireless network connection and one or more of the components of the computer 1601, such as the one or more processors 1603, a mass storage device 1604, an operating system 1605, VOC detection software 1606, sensing data 1607, a network adapter 1608, the system memory 1612, an Input/Output Interface 1610, a display adapter 1609, a display device 1611, and a human machine interface 1602, may be contained within one or more remote computing devices 1614A-1614C at physically separate locations, connected through buses of this form, in effect implementing a fully distributed system.

The computer 1601 may operate on and/or comprise a variety of computer-readable media (e.g., non-transitory). Computer-readable media may be any available media that is accessible by the computer 1601 and comprises non-transitory, volatile, and/or non-volatile media, removable and non-removable media. The system memory 1612 has computer-readable media in the form of volatile memory, such as random access memory (RAM), and/or non-volatile memory, such as read-only memory (ROM). The system memory 1612 may comprise data such as the sensing data 1607 and/or program modules such as the operating system 1605 and the VOC detection software 1606 that are accessible to and/or are operated on by the one or more processors 1603.

The computer 1601 may also comprise other removable/non-removable, volatile/non-volatile computer storage media. The mass storage device 1604 may provide non-volatile storage of computer code, computer-readable instructions, data structures, program modules, and other data for the computer 1601. The mass storage device 1604 may be a hard disk, a removable magnetic disk, a removable optical disk, magnetic cassettes or other magnetic storage devices, flash memory cards, CD-ROM, digital versatile disks (DVD) or other optical storage, random access memories (RAM), read-only memories (ROM), electrically erasable programmable read-only memory (EEPROM), and the like.

Any number of program modules may be stored on the mass storage device 1604, such as, by way of example, the operating system 1605 and the VOC detection software 1606. One or more of the operating system 1605 and the VOC detection software 1606 (or some combination thereof) may comprise elements of the programming and the VOC detection software 1606. The sensing data 1607 may also be stored on the mass storage device 1604. The sensing data 1607 may be stored in any of one or more databases known in the art. Examples of such databases comprise, DB2®, Microsoft® Access, Microsoft® SQL Server, Oracle®, mySQL, PostgreSQL, and the like. The databases may be centralized or distributed across multiple locations within the network 1615.

A user may enter commands and information into the computer 1601 via an input device (not shown). Such input devices may comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 1603 via a human-machine interface 1602 that is coupled to the bus 1613, but may be connected by other interface and bus structures, such as a parallel port, game port, an IEEE 1394 Port (also known as a Firewire port), a serial port, network adapter 1608, and/or a universal serial bus (USB).

A display device 1611 may also be connected to the bus 1613 via an interface, such as a display adapter 1609. It is contemplated that the computer 1601 may have more than one display adapter 1609 and the computer 1601 may have more than one display device 1611. A display device 1611 may be a monitor, an LCD (Liquid Crystal Display), light-emitting diode (LED) display, television, smart lens, smart glass, and/ or a projector. In addition to the display device 1611, other output peripheral devices may comprise components such as speakers (not shown) and a printer (not shown) which may be connected to the computer 1601 via Input/Output Interface 1610. Any step and/or result of the methods may be output (or caused to be output) in any form to an output device. Such output may be any form of visual representation, including, but not limited to, textual, graphical, animation, audio, tactile, and the like. The display 1611 and computer 1601 may be part of one device, or separate devices.

The computer 1601 may operate in a networked environment using logical connections to one or more remote computing devices 1614A-1614C. By way of example, a remote computing device 1614A-1614C may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computer 1601 and a remote computing device 1614A-1614C may be made via a network 1615, such as a local area network (LAN) and/or a general wide area network (WAN). Such network connections may be through the network adapter 1608. The network adapter 1608 may be implemented in both wired and wireless environments. Such networking environments are conventional and commonplace in dwellings, offices, enterprise-wide computer networks, intranets, and the Internet.

Application programs and other executable program components such as the operating system 1605 are illustrated herein as discrete blocks, although it is recognized that such programs and components may reside at various times in different storage components of the computing device 1601, and are executed by the one or more processors 1603 of the computer 1601. An implementation of the VOC detection software 1606 may be stored on or transmitted across some form of computer readable media. Any of the disclosed methods may be performed by computer readable instructions embodied on computer readable media. Computer readable media may be any available media that may be accessed by a computer. By way of example and not meant to be limiting, computer readable media may comprise “computer storage media” and “communications media.” “Computer storage media” may comprise volatile and non-volatile, removable and non-removable media implemented in any methods or technology for storage of information such as computer readable instructions, data structures, program modules, or other data. Exemplary computer storage media may comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by a computer.

The methods and systems may employ artificial intelligence (AI) techniques such as machine learning and iterative learning. Examples of such techniques comprise, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other compositions and methods for carrying out the several purposes of this disclosure. As a result, the claims should not be considered as limited by the description or examples.

What is claimed is:

1. A method comprising:
receiving, by a computing device, sensing data indicative of one or more voltammograms associated with a sample comprising one or more unknown analytes;
determining, based on the sensing data, a plurality of features associated with the one or more voltammograms, wherein the plurality of features are indicative of shapes or redox peaks associated with the one or more voltammograms;
determining, based on the plurality of features, one or more linear discriminants associated with the one or more unknown analytes; and
classifying, based on the one or more linear discriminants and one or more reference linear discriminants, the one or more unknown analytes, using a machine learning model configured to determine the one or more reference discriminants based on one or more known analytes.

2. The method of claim 1, wherein the one or more voltammograms are determined based on one or more cyclic voltammetry (CV) responses associated with the one or more unknown analytes.

3. The method of claim 1, wherein the plurality of features comprise one or more shape features and one or more redox peak features.

4. The method of claim 3, wherein the one or more shape features comprise one or more fitting parameters associated with the one or more voltammograms and one or more left-and-right endpoints of the one or more voltammograms.

5. The method of claim 3, wherein the one or more redox peak features comprise one or more peak heights associated with the one or more voltammograms, one or more peak areas associated with the one or more voltammograms, one or more peak potentials associated with the one or more voltammograms.

6. The method of claim 1, further comprising:
determining, based on the plurality of features, a linear diagram comprising the one or more linear discriminants and the one or more reference linear discriminants.

7. The method of claim 6, wherein the one or more linear discriminates are one or more data points in the linear diagram and the one or more reference linear discriminates are one or more reference data points in the linear diagram.

8. The method of claim 1, further comprising:
determining one or more projected means of the one or more reference linear discriminants associated with the one or more known analytes;
determining, based on the one or more linear discriminants and the one or more projected means of the one or more reference linear discriminants, one or more projected distances indicative of how close the one or more unknown analytes are to the one or more known analytes; and
determining, based on the one or more projected distances, the classification of the one or more unknown analytes.

9. The method of claim 1, wherein the one or more unknown analytes comprise a substance that is reactive with O₂⁻ in the ionic liquid (IL), a substance that has a different O₂ diffusion coefficient relative to the ionic liquid (IL), or both.

10. An apparatus comprising:
one or more processors; and
a memory storing processor-executable instructions that, when executed by the one or more processors, cause the apparatus to:
receive sensing data indicative of one or more voltammograms associated with a sample comprising one or more unknown analytes;
determine, based on the sensing data, a plurality of features associated with the one or more voltammograms, wherein the plurality of features are indicative of shapes or redox peaks associated with the one or more voltammograms;
determine, based on the plurality of features, one or more linear discriminants associated with the one or more unknown analytes; and
classify, based on the one or more linear discriminants and one or more reference linear discriminants, the one or more unknown analytes, using a machine learning model configured to determine the one or more reference discriminants based on one or more known analytes.

11. The apparatus of claim 10, wherein the plurality of features comprise one or more shape features and one or more redox peak features.

12. The apparatus of claim 11, wherein the one or more shape features comprise one or more fitting parameters associated with the one or more voltammograms and one or more left-and-right endpoints of the one or more voltammograms, and the one or more redox peak features comprise one or more peak heights associated with the one or more voltammograms, one or more peak areas associated with the one or more voltammograms, one or more peak potentials associated with the one or more voltammograms.

13. The apparatus of claim 10, wherein the processor-executable instructions that, when executed by the one or more processors, cause the apparatus to:
determine, based on the plurality of features, a linear diagram comprising the one or more linear discriminants and the one or more reference linear discriminants.

14. The apparatus of claim 13, wherein the one or more linear discriminates are one or more data points in the linear diagram and the one or more reference linear discriminates are one or more reference data points in the linear diagram.

15. The apparatus of claim 10, wherein the processor-executable instructions that, when executed by the one or more processors, cause the apparatus to:
determine one or more projected means of the one or more reference linear discriminants associated with the one or more known analytes;
determine, based on the one or more linear discriminants and the one or more projected means of the one or more reference linear discriminants, one or more projected distances indicative of how close the one or more unknown analytes are to the one or more known analytes; and
determine, based on the one or more projected distances, the classification of the one or more unknown analytes.

16. The apparatus of claim 10, wherein the one or more unknown analytes comprise a substance that is reactive with O₂⁻ in the ionic liquid (IL), a substance that has a different O₂ diffusion coefficient relative to the ionic liquid (IL), or both.

17. A system comprising:
a sensor device configured to:
acquire sensing data based on measurements of current density and potential versus counter and reference electrodes in a sample comprising an ionic liquid (IL), an aprotic solvent, and one or more unknown analytes; and
a computing device configured to:
receive, from the sensor device, the sensing data indicative of one or more voltammograms associated with the sample;
determine, based on the sensing data, a plurality of features associated with the one or more voltammograms, wherein the plurality of features are indicative of shapes or redox peaks associated with the one or more voltammograms;
determine, based on the plurality of features, one or more linear discriminants associated with the one or more unknown analytes; and
classify, based on the one or more linear discriminants and one or more reference linear discriminants, the one or more unknown analytes, using a machine learning model configured to determine the one or more reference discriminants based on one or more known analytes.

18. The system of claim 17, wherein the plurality of features comprise one or more shape features and one or more redox peak features.

19. The system of claim 17, wherein the computing device is further configured to:
determine one or more projected means of the one or more reference linear discriminants associated with the one or more known analytes;
determine, based on the one or more linear discriminants and the one or more projected means of the one or more reference linear discriminants, one or more projected distances indicative of how close the one or more unknown analytes are to the one or more known analytes; and
determine, based on the one or more projected distances, the classification of the one or more unknown analytes.

20. The system of claim 17, wherein the one or more unknown analytes comprise a substance that is reactive with O₂⁻ in the ionic liquid (IL), a substance that has a different O₂ diffusion coefficient relative to the ionic liquid (IL), or both.
